(19) United States
(12) Patent Application Publication
(10) Pub. No.: US 2026/0002869 A1
(43) Pub. Date: Jan. 1, 2026

(54) NEURODEGENERATIVE DISORDER BIOSENSING SYSTEM AND METHOD

(71) Applicant: ECOLE POLYTECHNIQUE FEDERALE DE LAUSANNE (EPFL), Lausanne (CH)

(72) Inventors: Deepthy KAVUNGAL, LAUSANNE (CH); Hatice ALTUG YANIK, MEX (CH); Hilal LASHUEL, ETOY (CH)

(21) Appl. No.: 18/756,559

(22) Filed: Jun. 27, 2024

(51) Int. Cl. G01N 21/3563; G01N 33/543; G01N 33/68

(52) U.S. Cl. CPC G01N 21/3563; G01N 33/54373; G01N 33/6896; G01N 2800/28

(57) ABSTRACT

A neurodegenerative disorder biosensing system including at least one plasmonic device including a plurality of plasmonic nanostructures; at least one optical detector; and at least one data processing device including at least one processor configured to process a plurality of absorption spectra determined from the reflected optical infra-red spectra, the plurality of absorption spectra representing time-resolved infra-red absorption by at least one of: (i) first protein secondary structure types formed from neurodegenerative disorder aggregated proteins and (ii) second protein secondary structure types formed from neurodegenerative disorder aggregated proteins. The at least one processor configured to process the plurality of absorption spectrum signals to identify the first protein secondary structure type and the second protein secondary structure type, and to distinguish the identified first protein secondary structure type from the second protein secondary structure type, and to distinguish the identified second protein secondary structure type from the first protein secondary structure type.

### FIELD OF THE INVENTION

The present invention relates to the field of neurodegenerative disorder biosensing and the detection of neurodegenerative disorder structural biomarkers, and more particularly to a neurodegenerative disorder biosensing system and a neurodegenerative disorder biosensing method for detecting neurodegenerative disorder structural biomarkers for diagnosis of neurodegenerative disorders, disease monitoring, and evaluation of therapies.

### BACKGROUND

Neurodegenerative diseases (NDDs) including Parkinson's disease (PD), Alzheimer's disease (AD), dementia with Lewy bodies, amyotrophic lateral sclerosis (ALS), and Huntington's disease are a set of heterogeneous disorders characterized by the progressive structural and functional degradation of the nerve cells and accumulation of misfolded and aggregated proteins in the affected brain regions. These disorders are increasing in prevalence, especially in aging societies, and pose huge economic and health burdens. Unfortunately, there are no effective treatments to prevent or slow the progression of these devastating diseases. Although most NDDs start 10 to 15 years before the manifestation of the clinical diagnostic symptoms, we still lack a reliable diagnostic method for early detection or monitoring of disease progression, thus precluding any effort for early intervention. Furthermore, NDDs are often misdiagnosed because of their clinical heterogeneity, and overlapping symptoms and brain pathologies. These observations underscore the need for reliable diagnostic biomarkers and techniques for the early detection of the NDDs, monitoring of disease progression, patient stratification, and investigation of the efficacy of new treatments and disease-modifying strategies.

Although distinct proteins are considered to be involved in different NDDs, protein misfolding into fibrillary aggregates via oligomeric species formation is found to be a common mechanism shared by NDDs and most proteinopathies.

For example, the pathological hallmark of PD is the presence of β sheet-rich aggregated species of the alpha-synuclein protein (aSyn; 14.5 kDa) in Lewy bodies (LBs) and Lewy neurites. Under physiological conditions, aSyn exists in equilibrium between a disordered conformation and α-helical—rich membrane-bound state. In pathological conditions, these two states undergo structural changes and self-assemble to form intermediary oligomers characterized by mixed secondary structure contents, which ultimately convert to β sheet-rich fibrillar aggregates. In the case of AD, similar structural changes are observed for the tau proteins and amyloid-beta peptides. Hence, identifying and detecting protein aggregates and their structural changes permit diagnosing NDDs and understanding their pathologies.

Recently, two drugs—aducanumab (Aduhelm) and lecanemab (Leqembi)—which are monoclonal antibodies preferentially targeting amyloid fibrils and oligomers, respectively, received accelerated Food and Drug Administration approval for early AD treatment. This not only shows the potential of the structural biomarkers to guide and assess future therapies but also, at the same time, underlines the importance of developing effective methods to detect and quantify the level and structural properties of different protein aggregates. Moreover, the compelling fact that these NDD-related proteins and their different structural forms are found in the body fluids like cerebrospinal fluid (CSF), blood, and saliva provide a path for creating minimally invasive detection tools based on the structural biomarker criteria.

Gold standard methods currently used for biomarker identification and quantification in NDD research, such as mass spectrometry (MS) and enzyme-linked immunosorbent assay (ELISA), focus on quantifying the level of target proteins but are insensitive to changes in their structural states and are thus not able to discriminate between different structural forms.

Progress in proteomic methods such as limited proteolysis-based MS (LiP-MS) shows promise in identifying even the structural alterations of proteins in complex samples on a global scale. However, these MS-based techniques are in the early stages of development, and integration of them into clinical practices remains challenging. ELISA, on the other hand, is an antibody-based detection method, and there are ongoing biochemical approaches focused on developing antibodies specific either to the oligomers or to the fibrils as a solution to structural insensitivity. But recent validation results indicate that none of the used antibodies are specific to only one form of aSyn aggregate or natively unstructured monomers.

Spectroscopic methods, including nuclear magnetic resonance and circular dichroism (CD), can probe the structural information of proteins and have been used to characterize NDD-related protein biomarkers. However, the need for laborious homogeneous sample preparations, the requirement of high protein concentration, and large sample volumes, as well as the inability to monitor the proteins in their physiological conditions, limit their use as a clinical diagnosis method. Recent advances have enabled the development of structural-based diagnostic assays such as protein misfolding cyclic amplification and real-time quaking-induced conversion, which are shown to differentiate patients with PD and AD from healthy individuals with more than 90% accuracy. However, they do not allow the assessment of the actual amounts of aggregates or the ratio of oligomers to fibrils in body fluids. Moreover, they lack the capability to enable prodromal diagnosis, monitor disease progression, and differentiate among the NDDs due to their overlapping pathologies. Incorporating multiple biomarkers associated with neuropathology and neurodegeneration could enable more accurate diagnostics.

Infrared (IR) spectroscopy is sought as a promising method for NDD diagnostics since it provides chemical-specific and structural-sensitive detection in a label-free and noninvasive manner. The chemical specificity of the IR technique has been used for the spectrochemical analysis of body fluids and tissues for PD and AD. Particularly, among the protein absorption bands, the amide I absorption band offers a unique ability to identify different secondary structure motifs like disordered, a helix, β sheets, and β turns. However, the low IR absorption cross section of the molecules and the overlap of water absorbance bands with that of proteins limit the use of the technique for conformational studies in the aqueous medium. Multiple reflection-based attenuated total reflection (ATR) overcomes these drawbacks to some extent. Recent works using this technique in combination with immunoassay showed promising results for the early detection of AD and ALS based on the secondary structural changes of amyloid-beta peptides and TDP-43 protein, respectively, present in body fluids.

Surface-enhanced IR absorption (SEIRA) spectroscopy is an emerging method to expand the biosensing capabilities of IR spectroscopy, see for example reference No. 44 listed herein further below, the disclosure of which is hereby incorporated herein by reference in its entirety for all purposes. In SEIRA, engineered nanostructures supporting strong and localized electromagnetic fields at their resonant wavelength can be designed to overlap spectrally with the vibrational bands of the biomolecules. This amplifies the absorption signals of immobilized biomolecules by orders of magnitude, even in the aqueous medium, via the plasmonic internal reflection (PIR) effect, see for example reference No. 46 listed herein further below, the disclosure of which is hereby incorporated herein by reference in its entirety for all purposes.

Earlier sensing applications have focused on detecting only the presence of proteins in a dry medium, and some studies extended its use to secondary structure analysis. Integration of SEIRA with microfluidics has advanced the capabilities to conduct in situ and in-flow experiments for extracting biomolecular interaction kinetics in real time. This also facilitated the protein sensing to be done in situ by directly capturing proteins on the chip surface for sensing and secondary structure identification and even for the real-time monitoring of protein secondary structure changes, but in a nonspecific manner. Despite these recent progresses, SEIRA has not been shown to decode the structural information of proteins from disease-related biomarkers in pre-clinical level/for real-world applications. For such applications, it is additionally desirable that the sensing method is highly specific to the target biomarkers from a small sample volume in a complex biomatrix, can distinguish between the pathological aggregates, and monitor a panel of complementary biomarkers.

Therefore, there is a need for a neurodegenerative disorder biosensing system and a neurodegenerative disorder biosensing method to improve detecting and distinguishing neurodegenerative disorder structural information of disease-related protein biomarkers, that may additionally be highly specific to the target structural biomarkers from a small sample volume in a complex biomatrix, that may additionally be able to distinguish between pathological aggregates, and that may be able to monitor a panel of complementary biomarkers. There is also a need for a neurodegenerative disorder biosensing system and a neurodegenerative disorder biosensing method to diagnosis neurodegenerative disorders, to monitor such diseases, and to evaluate therapies.

### SUMMARY

According to one aspect of the present invention, a neurodegenerative disorder biosensing system is provided. The system may include at least one plasmonic device including a plurality of plasmonic nanostructures configured to provide plasmonic excitation surface-enhanced infra-red absorption by molecular vibrational excitations of neurodegenerative disorder proteins, the plurality of plasmonic nanostructures being configured to have attached thereto capturing agents configured to bind to protein secondary structure types formed from neurodegenerative disorder aggregated proteins; at least one optical detector configured to detect reflected optical infra-red spectra reflected from the plurality of plasmonic nanostructures of the at least one plasmonic device; and at least one data processing device configured to process a plurality of absorption spectra determined from the reflected optical infra-red spectra, the plurality of absorption spectra representing time-resolved infra-red absorption by at least one of: (i) first protein secondary structure types formed from neurodegenerative disorder aggregated proteins and (ii) second protein secondary structure types formed from neurodegenerative disorder aggregated proteins. The at least one data processing device may be configured to process the plurality of absorption spectrum signals to identify the first protein secondary structure type and the second protein secondary structure type, and to distinguish the identified first protein secondary structure type from the second protein secondary structure type, and to distinguish the identified second protein secondary structure type from the first protein secondary structure type, wherein the first protein secondary structure type is different to the second protein secondary structure type.

According to another aspect of the present invention, a neurodegenerative disorder biosensing method is provided. The method may include:
providing at least one plasmonic device including a plurality of plasmonic nanostructures configured to provide plasmonic excitation surface-enhanced infra-red absorption by molecular vibrational excitations of neurodegenerative disorder proteins, the plurality of plasmonic nanostructures having attached thereto capturing agents configured to bind to protein secondary structure types formed from neurodegenerative disorder aggregated proteins;
providing at least one optical detector configured to detect reflected optical infra-red spectra reflected from the plurality of plasmonic nanostructures of the at least one plasmonic device;
providing at least one fluidic sample to the at least one plasmonic device;
determining a plurality of absorption spectra from obtained reflected optical infra-red spectra, the plurality of absorption spectra representing time-resolved infra-red absorption by at least one of: (i) first protein secondary structure types formed from neurodegenerative disorder aggregated proteins and (ii) second protein secondary structure types formed from neurodegenerative disorder aggregated proteins; identifying, from the plurality of absorption spectra, at least one the first protein secondary structure type and the second protein secondary structure type, and distinguishing the identified first protein secondary structure type from the second protein secondary structure type and the identified second protein secondary structure type from the first protein secondary structure type, wherein the first protein secondary structure type is different to the second protein secondary structure type.

According to still another aspect of the present invention, a non-transitory computer-readable medium is provided, having computer instructions recorded thereon. The computer code is configured to perform the neurodegenerative disorder biosensing method when executed on a data processing device of a computer device.

The above and other objects, features and advantages of the present invention and the manner of realizing them will become more apparent, and the invention itself will best be understood from a study of the following description and appended claims with reference to the attached drawings showing some preferred embodiments of the invention.

### BRIEF DESCRIPTION OF THE SEVERAL DRAWINGS

The accompanying drawings, which are incorporated herein and constitute part of this specification, illustrate the presently preferred embodiments of the invention, and together with the general description given above and the detailed description given below, serve to explain features of the invention.

FIG. 1 schematically shows an exemplary embodiment of a neurodegenerative disorder biosensing system according to the present disclosure.

FIG. 2A shows a misfolding pathway of the alpha-synuclein (aSyn) protein showing the transition from unstructured monomers to β sheet-enriched fibrillar species via a heterogenous population of oligomers.

FIG. 2B is a schematic side view of part of an exemplary embodiment of a neurodegenerative disorder biosensing system according to the present disclosure showing an exemplary optofluidic setup used for backside-reflected Surface-enhanced IR absorption (SEIRA) measurements and shows the capture of all aSyn structural species by antibodies used to form an immunoassay coupled optofluidic Surface-enhanced IR absorption (ImmunoSEIRA).

FIG. 2C shows amide I and amide II absorption bands extracted from the captured proteins with a plasmonically enhanced SEIRA signal. Within the amide I band, contributions from different structural motifs like β sheets, disordered, and β turns are identified to extract structural information.

FIG. 3A is an image of an embodiment of a nanoplasmonic sensor or device included on an infrared transparent substrate with an exemplary three-row microarray design. Each row includes gold mirrors and sensing elements. For example, the top row contains three gold mirrors at the two ends and the center and four sensing elements between them. Scanning electron microscopy images show a part of the sensor element with periodically arranged unit cells consisting of plasmonic nanorods designed with exemplary dimensions of L=1.5 μm, G=0.08 μm, and Py=3.2 μm.

FIG. 3B shows an image of an exemplary chipcell fabricated according to the substrate shape to hold the nanoplasmonic sensor or chip, and a micro-flowcell, including a plurality of microfluidic channels, that follows the three-row design of the sensor.

FIG. 3C shows characterization of the nanorod arrays using a simulation solver and compared with a measurement taken in an aqueous medium using a Fourier transform infrared spectrometer to observe the resonance targeting the amide II and I bands around 1500 to 1700 cm⁻¹. a.u., arbitrary units.

FIG. 3D shows in vitro synthesized pure species of aSyn monomers, oligomers, and fibrils (in the order from top to bottom) characterized by transmission electron microscopy showing the differences in their morphologies.

FIG. 4A schematically shows a three-step immunoassay used in an exemplary ImmunoSEIRA assay for the capture and spectral analysis of aSyn species. The sensor chip is incubated with N-hydroxysuccinimide-activated carboxyl thiols and spacer OH thiols overnight. Then, with the optofluidic configuration, the SYN-211 antibody is injected in situ and washed with the buffer to form a stable antibody layer. Last, the aSyn fibrils are injected and washed to selectively enrich the target on the functionalized sensor surface.

FIG. 4B shows, from time-resolved reflectance measurements taken for each step starting from the thiol baseline, the integrated absorbance signal over the amide spectral region which is used to extract a sensorgram, which shows the injection, binding, and stabilization of antibody as well as the successful capture of aSyn fibrils along with subsequent washing steps.

FIG. 4C shows absorbance spectrum as a function of time for each assay step. The distinct absorption signatures of the antibody and aSyn fibrils in the amide range can be visualized.

FIG. 4D (left) shows that time-resolved reflectance measurements are taken for each step starting from the antibody baseline, and the integrated absorbance signal over the amide region is used to extract a sensorgram, which shows the in-flow injection, binding, and stabilization of the aSyn oligomers along with subsequent washing step. For clarity and better visualization of the aSyn oligomer signature, the data is shown from the stabilized antibody-bound surface taken as the reference. FIG. 4D (right) shows absorbance spectra as a function of time for the oligomer binding. The distinct absorption signatures of the aSyn oligomers in the Amide range can be visualized.

FIG. 4E (left) shows that time-resolved reflectance measurements are taken for each step starting from the antibody baseline, and the integrated absorbance signal over the amide region is used to extract a sensorgram, showing the in-flow injection, binding, and stabilization of the aSyn monomers, along with the subsequent washing step. For clarity and better visualization of the aSyn monomer signature, the data is shown from the stabilized antibody-bound surface taken as the reference. FIG. 4E (right) shows absorbance spectra as a function of time for the monomer binding. The distinct absorption signatures of the aSyn monomers in the Amide range can be visualized.

FIG. 4F concerns two in-flow experiments involving the injection of a same amount of SYN-211 antibody (200 μl, 40 μg/ml) over the thiolated sensor surfaces. FIG. 4F (top left) that in the first experiment, after the antibody immobilization step ˜7 μM of aSyn fibrils is injected without blocking and one observes the binding of the fibrils with respect to the baseline of antibody immobilized surface as the reference. The corresponding sensorgram registers an overall increase of integrated absorbance of 1 mODcm⁻¹ upon fibrils binding. FIG. 4F (top right) shows the second experiment in which a blocking step is executed by injecting 300 μl of milk buffer (2×) before the same amount of aSyn fibrils injection as in the first experiment. Here also, the overall increase of sensorgram signal upon fibrils binding, with the blocking step as a reference, is 1 mODcm⁻¹. To further verify the similar level of aSyn binding response in both scenarios, the time-resolved absorbance of aSyn fibrils is shown. FIG. 4F (bottom left) shows the measured aSyn fibrils absorbance without blocking and FIG. 4F (bottom right) shows that this is similar to the case with blocking.

FIG. 5A shows the original absorption spectra of the different aSyn structural forms, from left to right, Monomers, Oligomers, and Fibrils that were used for the deconvolution analysis.

FIG. 5B shows conformational profiling of different aSyn species. Each aSyn pure structural species, monomers, oligomers, and fibrils, are captured separately on different chips that are identically functionalized. A second-derivative analysis is used to identify the main peak contributions, and correspondingly, Fourier self-deconvolution (FSD) and curve fitting are done to identify contributions from individual structural motifs. FIG. 5B (left) shows that aSyn monomers have characteristic disordered structures evident from almost 70% absorption from those bands, with only 20% of β sheets present. FIG. 5B (middle) shows that aSyn oligomers have 50% of their structure in β sheet configuration, and the rest are almost equally present as β turns and disordered states. FIG. 5B (right) shows that aSyn fibrils have the highest contribution from β sheets—58% among all species, with disordered motifs contributing 27% and the rest β turns.

FIG. 6A shows a plausible scheme of total aggregate presence in a sample at different stages of a disease with mostly oligomeric aSyn in the beginning but with the gradual increase of fibrils presence and eventually becoming the dominant species in the mixture for AI-aided ImmunoSEIRA for accurate prediction of a quantitative presence of oligomers and fibrils distinctively from mixed samples.

FIG. 6B shows a bar plot of the titrated concentration mixes of oligomers and fibrils used.

FIG. 6C shows three-dimensional spectrograms (Absorbance-Wavenumber-Timepoints) of all the mentioned mixtures that are collected using the ImmunoSEIRA setup of the present disclosure and labeled according to the presence of fibrils in each sample set.

FIG. 6D shows a comparison of the actual fibrils percentage with the predicted value by the deep neural network (DNN) from the testing data showing excellent accuracy.

FIG. 6E shows a sample dataset with labels for AI analysis, and shows the molar percentages of aSyn oligomers and fibrils present in the mixed samples used for DNN analysis and their respective label fed to the DNN.

FIG. 6F (top) illustrates a surface functionalization scheme used in measurements. FIG. 6F (top-right) shows a bar plot of two new titrated concentration mixtures of aSyn oligomers and fibrils spiked in human CSF, corresponding to (90% oligomers & 10% fibrils and 10% oligomers & 90% fibrils) at a fixed final concentration of 5 μM. FIG. 6F (middle) shows 3D spectrograms (Absorbance-Wavenumber-Timepoints) of two new mixtures that are concatenated with their corresponding labels according to the presence of fibrils (Label=10 for 90% oligomers & 10% fibrils mixture and Label=90 for 10% oligomers & 90% fibrils mixture) in each sample set. Both datasets are then split into 80-20%, respectively. After training with 80% data, the remaining 20% of testing data is fed to the newly trained network to predict the percentage presence of oligomers and fibrils in spiked CSF. FIG. 6F (bottom) shows the results predicted by the trained DNN for these new datasets to get a comprehensive visualization of the AI-aided quantitative distinction of aSyn aggregates using ImmunoSEIRA. The comparison of the actual fibrils percentage with the predicted value by DNN from the testing data shows a good correlation with an accuracy of 92.31%. The AI-aided analysis successfully fared well in decoupling even the close-by percentages: 0-100, 10-90, 25-75% (also 100-0, 90-10, 75-25%).

FIG. 7A concerns a cross-reactivity experiment of the multiplexed ImmunoSEIRA for aSyn or tau fibrils injection and is a schematic of the in-flow experiment showing blocking buffer injection followed by the aSyn or tau fibril injection in two separate experiments. FIG. 7B shows that in the first experiment, the sensorgram obtained from the SYN-211 printed sensing element shows a permanent signal increase after the final washing step due to the specific binding of aSyn fibrils on the SYN-211 antibody, whereas the sensorgram from HT7-printed sensing element shows that the signal levels before aSyn fibril injection and after the final washing steps are similar, indicating no cross-reactivity of HT7 antibody to aSyn fibrils. FIG. 7C shows the retrieved absorbance (between 85 and 155 min) from SYN-211 showing the corresponding spectral signal increase and FIG. 7D shows the absorbance retrieved from HT7 for this relevant time window (between 85 and 155 min) showing no spectral signal increase.

FIG. 7E shows a second measurement with tau fibrils injection, and the sensorgram obtained from SYN-211-printed sensor element showing that the signal levels before tau fibril injection and after the final washing steps are similar, indicating no cross-reactivity of the SYN-211 antibody to tau fibrils, but the sensorgram obtained from HT7-printed sensor element shows a permanent signal increase after the final washing step due to the specific binding of tau fibrils on HT7 antibodies.

FIG. 7F shows the retrieved absorbance (between 80 and 150 min) and shows no stable spectral signal increase for the SYN-211 sensing element.

FIG. 7G shows the absorbance retrieved from the HT7 printed sensing element for the same selected time window as that of FIG. 7F showing the corresponding spectral signal increase by the binding of tau protein.

FIG. 8A concerns a multiplexed ImmunoSEIRA demonstration with simultaneous injection of aSyn and tau fibrils is a schematic of the in-flow experiment showing a blocking buffer injection followed by the injection of a mixed sample containing both aSyn and tau fibrils.

FIG. 8B shows a stable binding of aSyn fibrils on the SYN-211-printed sensing element during the mixture injection and the final washing step is indicated by the consistent signal increase in the sensorgram curve.

FIG. 8C similarly shows stable binding of tau fibrils on the HT7-printed sensing element during the mixture injection and the final washing step is indicated by the consistent signal increase in the sensorgram.

FIG. 8D shows the retrieved time-resolved absorption spectra (between 90 and 230 min) from the SYN-211 sensing element showing a corresponding spectral increase by the binding of aSyn protein.

FIG. 8E shows the absorbance retrieved from the HT7 printed sensing element showing a spectral increase for the same time window as that of FIG. 8D due to the specific binding of tau protein.

FIG. 9A concerns obtaining an absorption signature of pathological aSyn fibrils that can be accurately retrieved in the presence of complex biomatrix and is a schematic of the assay steps for characterizing aSyn fibrils binding in human CSF biomatrix with in-flow SEIRA measurements.

FIG. 9B shows the sensorgram highlights of different stages of binding. During the injection of aSyn-depleted human CSF, the signal increases due to nonspecific biomolecule presence. But after the washing step, the signal goes back to the previous baseline, indicating that the surface blocking is successful. The signal level after the injection of aSyn fibrils spiked human CSF and the final washing step increased, indicating that protein binds to the surface even in the presence of a complex biomatrix.

FIG. 9C shows that the characteristic absorbance fingerprints of the aSyn fibrils are accurately retrieved from the time-resolved absorbance spectra of the final step of spiked injection.

FIG. 10 (top) is a schematic of an in-flow experiment shows blocking buffer injection followed by the injection of a mixed sample containing aSyn aggregates (oligomers and fibrils in 25-75% ratio, respectively) and tau fibrils in human CSF. The concentration in 200 μl of injected CSF amounts to 6 μM for Tau fibrils and 5 μM for aSyn aggregate mixture. FIG. 10 (middle left) shows, after the blocking and washing steps, that a stable binding response is observed for the SYN-211 printed sensing element upon the sample injection (˜190 min), even after the final washing step. FIG. 10 (bottom left) shows the retrieved time-resolved absorption spectra of the sample on the SYN-211 array capturing the distinct signatures of aSyn aggregate mixture. FIG. 10 (middle right) similarly shows that a stable binding response for the HT7 printed sensing element upon the sample injection (˜190 min) is observed even after the final washing step. FIG. 10 (bottom right) shows the retrieved time-resolved absorption spectra of the HT7 array showing the distinct signatures of tau fibrils, thereby successfully demonstrating multiplexed ImmunoSEIRA in the complex matrix with aggregated protein mixtures.

FIGS. 11A and 11B show images of an embodiment of a microfluidic device of the neurodegenerative disorder biosensing system of the present disclosure, the microfluidic device including a plurality of microwells. Each microwell can be associated with a sensing area of a plasmonic device to capture neurodegenerative disorder proteins present in the microwell. This enables simultaneous sensing of a plurality of different samples, for example up to 96 different samples, in a fast, label-free and high-throughput manner using very small sample volumes, highly desired specifications for drug screening assays. This enables simultaneous sensing of same protein conformation incubated with different drugs, or different structural forms with same drug and/or different concentration gradients.

FIG. 11C is a schematic side view of a microfluidic device and a plasmonic device attached thereto or received thereby, the plasmonic device including a plurality of microwells associated with a sensing area of a plasmonic device to capture neurodegenerative disorder proteins present in the microwell.

FIG. 11D schematically shows a top perspective view of an exemplary plasmonic device including a plurality of microwells, where each microwell incudes a sensing area or element comprising a plurality of plasmonic nanostructures or nanoantennas. FIG. 11D also shows protein bioprinting of the plasmonic nanostructures or nanoantennas contained in the microwell.

FIG. 11E schematically shows an exemplary fabrication process of an exemplary plasmonic device including a plurality of microwells, where each microwell incudes at least one or a plurality of plasmonic nanostructures or nanoantennas.

FIG. 12A shows the plasmonic resonance of a sensing element tuned to resonate around 1600 cm⁻¹ in wet conditions (‘Reference’, R₀) and the resonance spectrum collected after aSyn fibrils have bound to thiols on the sensor surface (‘Fibrils binding’, R_{NDD}). The spectral shift in FIG. 12A, as well as the dips in the reflection spectrum of the latter, indicates the successful binding of aSyn fibrils on the surface. FIG. 12B shows the differential absorbance (mOD) of the aSyn fibrils calculated as −1000*log 10 (R_{NDD}/R0), and the obtained result is shown. The asymmetric line profile obtained for the protein absorption bands is clearly visible. As seen in FIG. 12C, one can then perform a baseline correction to get a final absorption spectra, which are shown as the results in the figures in the main text and the supplementary information. The obtained absorption spectra were fitted with a second-order polynomial (lower curve). FIG. 12D shows, after subtracting the polynomial from the spectra, the retrieved baseline-corrected absorbance spectra, which show vibrational features as known from conventional IR spectroscopy.

Herein, identical reference numerals are used, where possible, to designate identical elements that are common of the figures. Also, the images in the drawings are simplified for illustration purposes and may not be depicted to scale.

### DETAILED DESCRIPTION OF THE SEVERAL EMBODIMENTS

FIG. 1 schematically shows an exemplary embodiment of a neurodegenerative disorder biosensing system or device 1 according to an embodiment of the present disclosure. The neurodegenerative disorder (NDD) biosensing system 1 may include at least one plasmonic device or chip 3, at least one optical detector 5 and at least one light source 7 for providing light to the plasmonic device 3 for reflection measurements therefrom, the reflected light being provided to the optical detector 5.

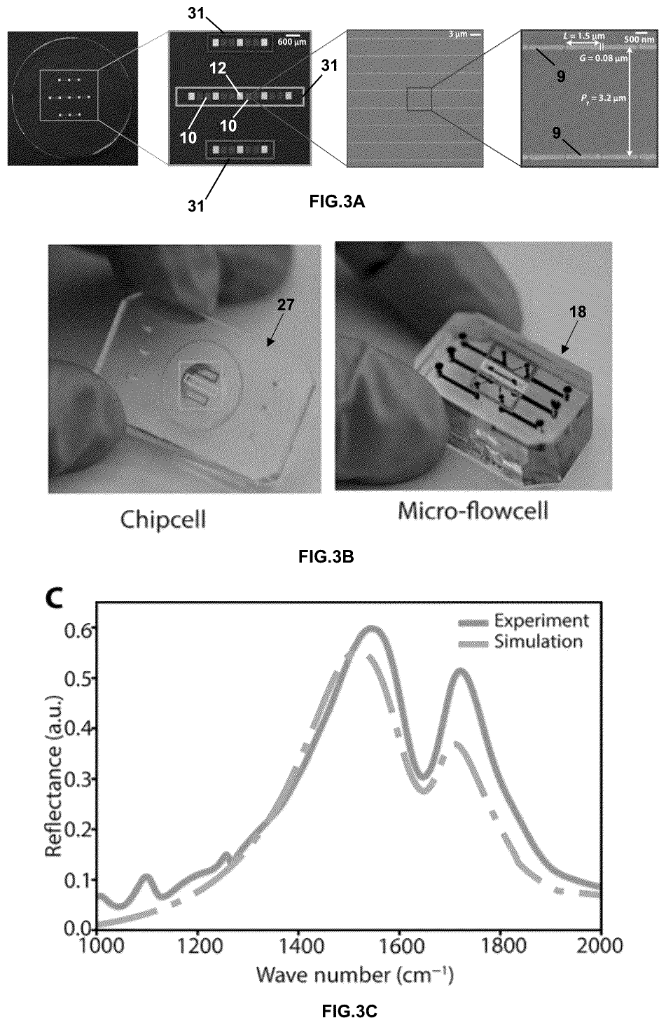

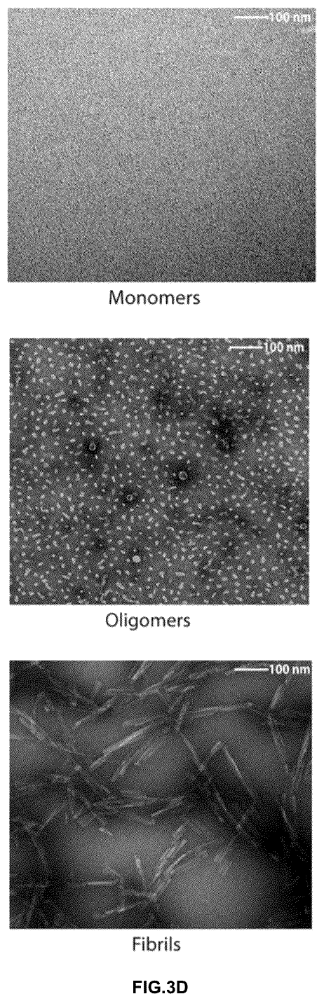

The plasmonic device 3 includes a plurality of plasmonic nanostructures or nanoantennas 9. The plurality of plasmonic nanostructures or nanoantennas 9 may form or define at least one sensing element or sensing area 10 of the plasmonic device 3. The plasmonic device 3 may include a plurality of sensing elements 10, as for example, shown in FIG. 3A where multiple sensing elements 10 are shown separated by mirrors 12 in this exemplary illustrated embodiment.

The plasmonic nanostructures 9 are configured and arranged so as to enhance light absorption by molecules that attach to or are captured by the plasmonic nanostructures 9. The plasmonic nanostructures 9 are configured to generate surface plasmons, collective oscillations of electrons, when resonantly excited by incident light. This generates large electromagnetic fields. Infrared vibrations of molecules located in these large electromagnetic fields are significantly enhanced resulting in an enhanced light absorption by these molecules.

The plasmonic nanostructures 9 are configured to provide plasmonic excitation surface-enhanced infra-red (IR) absorption by or of molecular vibrational excitations of neurodegenerative disorder (NDD) proteins. The plasmonic nanostructures 9 are configured to enhance the IR absorption of the molecules of the neurodegenerative disorder proteins.

Each nanostructure 9 preferably includes at least one metal or noble metal, such as gold, silver, copper, titanium, palladium, or aluminium; or consists essentially of a metal or noble metal, such as gold, silver, copper, titanium, palladium, or aluminium.

The plurality of nanostructures 9 preferably form a sensing element 10 including at least one or a plurality of arrays of nanostructures 9. In a preferred exemplary embodiment, the array is a substantially linear array nanostructures 9 extending across the plasmonic device 3, the sensing element 10 including a plurality of such arrays each separated by a distance P_{y}. The nanostructures 9 of the array are separated by a separation distance G with the nanostructure 9 having a length L. The separation distance P_{y} is greater than the nanostructure length L which is greater than the separation distance G.

The array separation P_{y}, the nanostructure 9 dimensions (length L) and the location of the nanostructure 9 (separation distance G) from a directly adjacent or neighboring nanostructure 9 are set or determined so that the plasmonic resonance of the nanostructures 9 and the sensing element 10 resonates at a wavelength or wavenumber that overlaps spectrally or corresponds spectrally with or to vibrational bands of the biomolecules of the neurodegenerative disorder proteins, permitting to enhance the IR absorption of these molecules.

In a preferred embodiment, the nanostructure 9 have an elongated structure or form, and the array forms a periodic array.

As a result, the nanostructures 9 support strong and localized electromagnetic fields at their resonant wavelength that is designed to overlap spectrally with the vibrational bands of the biomolecules of the neurodegenerative disorder proteins. This amplifies the absorption signals of immobilized biomolecules on the nanostructures 9 even in the aqueous medium, via the plasmonic internal reflection (PIR) effect.

The plurality of nanostructures 9 preferably form a plurality of arrays, each array extending across the plasmonic device 3. The plurality of nanostructures 9 of an array, for example, extend substantially linearly. Directly adjacent or neighboring arrays are separated by an array separation distance P_{y} (see, for example, FIG. 3A). The sensing element of the plasmonic device 3 may thus include a plurality of arrays nanostructures 9.

In an alternative embodiment, the plurality of nanostructures 9 may comprise a plurality of dielectric nanostructures. The dielectric nanostructures or dielectric metasurfaces are for example periodically arranged nanostructures on a support or substrate, and include or consist essentially of high-refractive index materials such as Ge, Si, GaAs, TiO2, GaN, Si₃N₄, provided here as non-limiting examples of high refractive index materials.

The resonance mechanisms in all-dielectric metasurfaces are governed by the interplay between the structured dielectric materials and the incident electromagnetic waves. These resonances can be excited and controlled through careful design of the metasurface geometry (size, shape, orientation, breaking symmetry), choice of materials (refractive index and absorption losses), and tuning of the incident light properties (wavelength, polarization, and angle of incidence), leading to significant enhancements in the local electromagnetic fields and enabling a wide range of optical and sensing applications.

The resonance excitation of dielectric metasurfaces have different mechanism compared to the plasmonics and based on the abovementioned parameters, it could be either via: 1) Mie resonances that occur due to the scattering of light by periodically arranged high-refractive-index dielectric nanoparticles/nanostructures and when the size of the nanostructures become comparable to the wavelength of the incident light, they support both electric and magnetic dipole resonances (as well as higher order quadrapole, octupole modes), leading to significant enhancement of the local electromagnetic fields, or through 2) Bound States in the Continuum (BIC), which are special states where the resonant modes are theoretically bound and do not radiate energy into the far field. However, by slightly perturbing the system (e.g., by introducing asymmetry or varying incident light angle), these bound states can be transformed into quasi-BICs with very high but finite quality factors (Q). These quasi-BICs result in sharp resonances with extremely high field enhancement due to the near-zero radiation losses. This phenomenon occurs due to the destructive interference of different radiation channels, leading to minimal leakage of energy.

Due to the material property and resonance mechanism, dielectric metasurfaces offers high Quality factor, low intrinsic loss, CMOS compatibility etc.

The plasmonic device 3 includes a substrate or support 11. The substrate or support 11 is comprised of a material that is transparent to the measurement light provided or emitted by the light source 7. The substrate or support 11 is preferably comprised of a material that is transparent to the infra-red light in the absorbing wavelength range of the biomolecules of the neurodegenerative disorder proteins. For example, the substrate or support 11 includes or consists essentially of CaF₂. This permits measurement light provided by the light source 7 to pass through the support 11 and the plasmonic device 3 to a protein sensing side of the plasmonic device 3 and to reflect light back through the support 11 and the plasmonic device 3 to the optical detector 5.

The plurality of nanostructures 9 are attached to the protein sensing side of the support 11 and may each be attached thereto by an adhesion element such as a Cr layer or a titanium (Ti) layer that has a thickness significant smaller than that of the nanostructure 9. The protein sensing side is located on an opposite side to a light incident side of the support 11 upon which measurement light is incident from the light source 7.

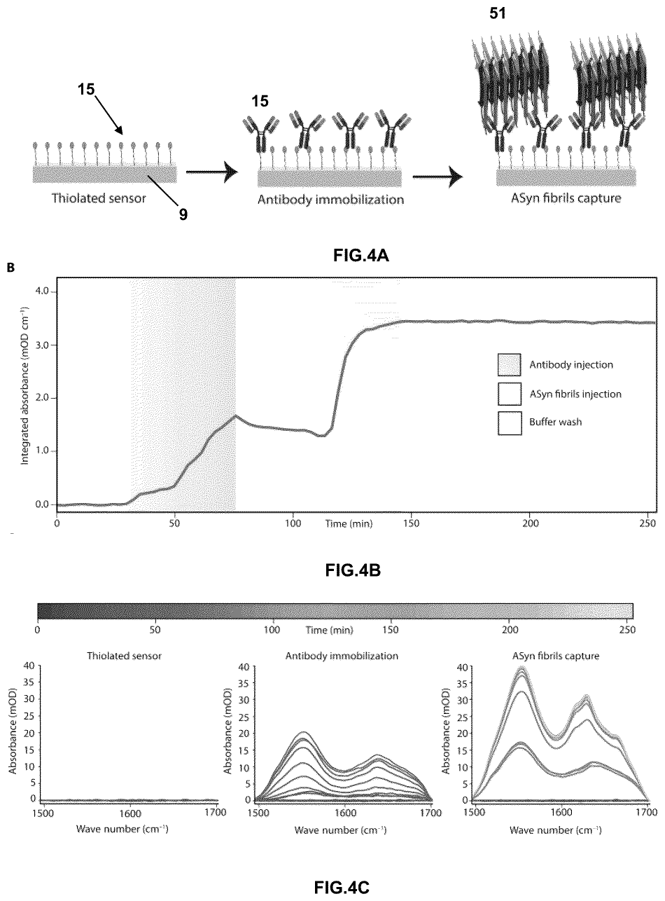

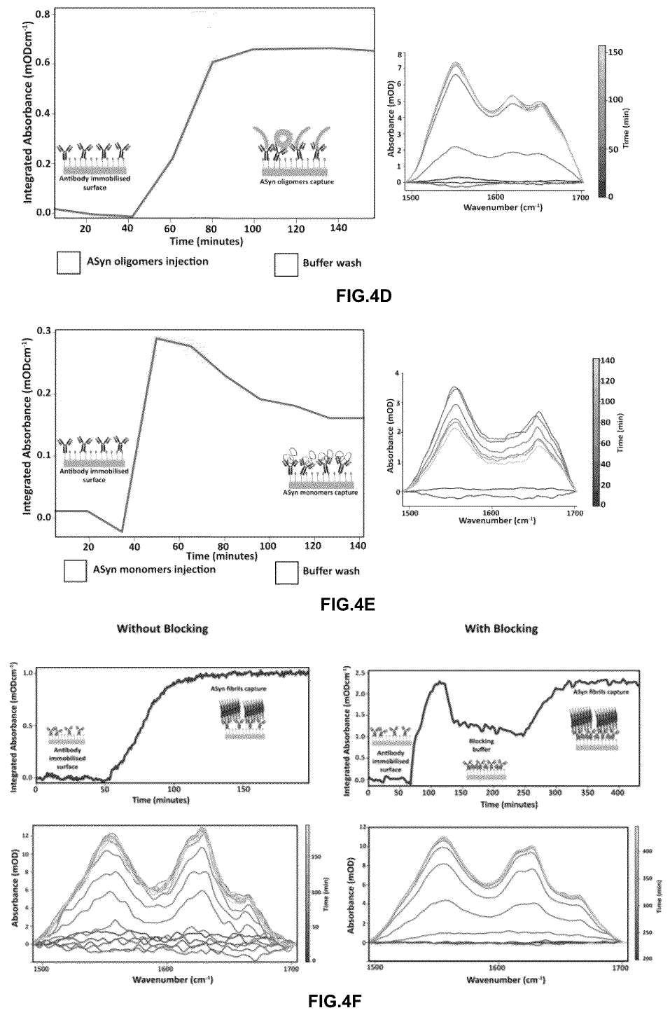

The plasmonic nanostructures 9 can be functionalized, that is, to have capturing agents 15 (see for example, FIG. 4A) attached to the plasmonic nanostructure 9. The capturing agent 15 is preferably configured to bind to neurodegenerative disorder monomeric proteins and to protein secondary structure types formed from neurodegenerative disorder aggregated proteins. For example, as described further below, for the capture and spectral analysis of aSyn species, the capturing agent 15 may include or consists essentially of a SYN-211 antibody attached to the plasmonic nanostructures 9 by thiols.

The neurodegenerative disorder biosensing system 1 may also include at least one fluidic device 17, for example, a microfluidic device 17. The microfluidic device 17 is configured to receive and support the plasmonic device 3 for biosensing to be performed on a sample, for example, a liquid sample provided to the plasmonic device 3 through the microfluidic device 17.

The microfluidic device 17 may include at least one fluid flow cell or fluid flow device 18 through which a fluid or liquid sample is communicated and at least one support device or chipcell 27 (see, for example, FIG. 1). The support device or chipcell 27 may include the plasmonic device 3, and the support device or chipcell 27 is configured to be attached to the flow cell or device 18 so as to position the plasmonic nanostructures 9 of the plasmonic device 3 in contact with the liquid sample to permit capture of NDD proteins present in the liquid sample.

The microfluidic device 17 and/or the flow cell 18 may include at least one or a plurality of fluidic channels 19, for example microfluidic channels 19, through which at least one liquid fluid is provided or flowed to the plasmonic device 3. Liquid fluid or a sample may be provided directly to the microfluidic channel 19, for example, using a dispenser. The microfluidic device 17 may include at least one inlet 21 in fluid communication with the microfluidic channel 19 and configured to introduce a liquid sample to the microfluidic channel 19 for transport to the plurality of plasmonic nanostructures 9 of the plasmonic device 3. The microfluidic device 17 is, for example, configured to communicate at least one fluid or liquid sample to the plurality of plasmonic nanostructures 9 from the fluid inlet 21 for immunoassay measurements. The microfluidic device 17 may include at least one outlet 23 in fluid communication with the microfluidic channel 19 and configured to remove or transport the liquid sample away from the plurality of plasmonic nanostructures 9 of the plasmonic device 3, for example, out of the microfluidic device 17. The inlet 21 can be connected to an external pump coupled with a flow rate sensor to precisely control the sample or liquid flow rate through the microfluidic channel 19.

The microfluidic device 17 may include an aperture or passage 25 exposing at least one portion of the at least one microfluidic channel 19. The aperture 25 is configured to receive the plurality of plasmonic nanostructures 9 of the plasmonic device 3 so as to locate the plurality of plasmonic nanostructures 9 or the sensing element 10 in fluidic contact with the liquid sample, or adjacent to or inside the fluidic channel 19 to permit capture of NDD proteins present in the liquid sample. The aperture or passage 25 may, for example, be configured to receive at least a portion of the plasmonic device 3 so as to locate the plurality of plasmonic nanostructures 9 adjacent to or inside the fluidic channel 19 to permit capture of NDD proteins present in the liquid sample (see exemplary embodiment of FIG. 1).

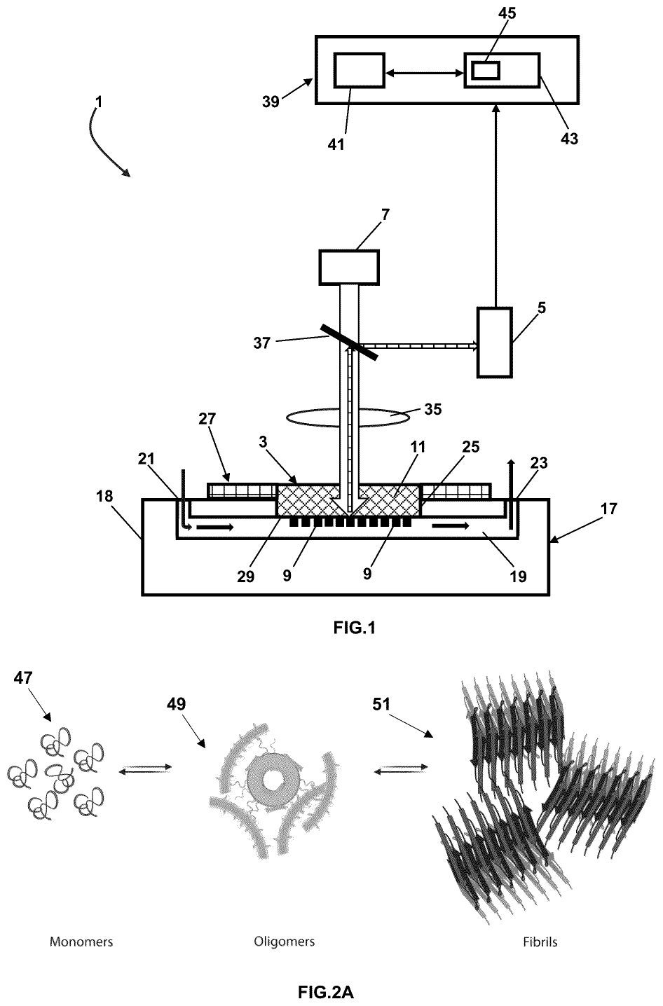

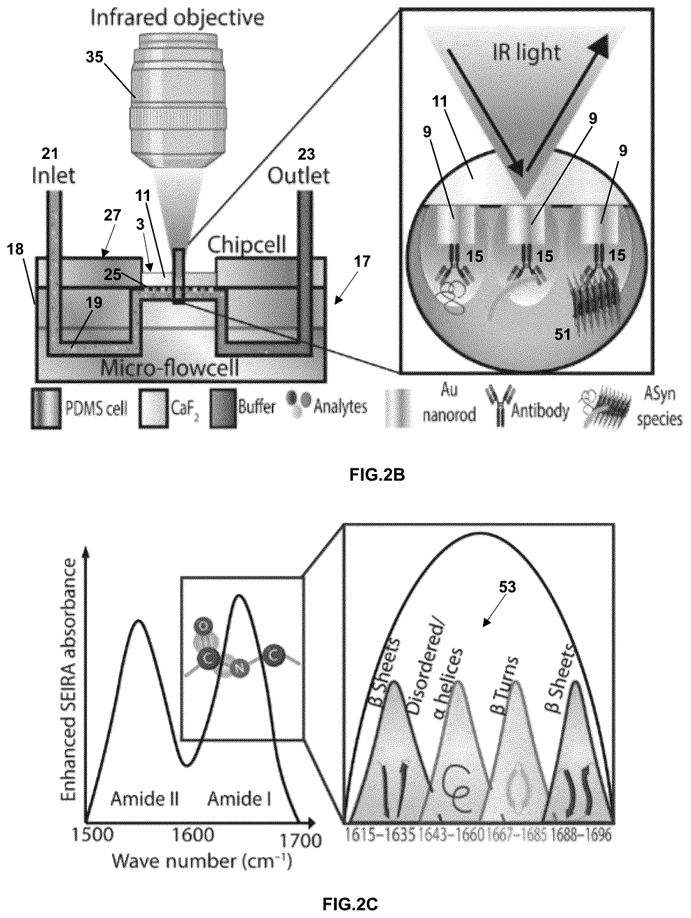

Alternatively, the aperture 25 may permit the plurality of plasmonic nanostructures 9 of the plasmonic device 3 to be located in the fluidic channel 19 when the plasmonic device 3 is positioned on the microfluidic device 17 (see exemplary embodiment of FIG. 2B), for example, when the support device or chipcell 27 is attached to the flow cell or device 18 so as to position the plasmonic nanostructures 9 of the plasmonic device 3 in contact with the liquid sample to permit capture of NDD proteins present in the liquid sample.

The microfluidic device 17 may include the plasmonic device 3 or be configured to receive the plasmonic device 3 such that at least a portion 29 of the plasmonic device 3 defines at least a portion of the at least one microfluidic channel 19. The plasmonic device 3 closes an exposed or open portion of the microfluidic channel 19 and places the plasmonic nanostructures 9 in fluidic contact with the liquid sample when a liquid sample is present in the microfluidic channel 19.

The microfluidic device 17 can be displaced relative to the objective lens 35, by for example, a translational stage, to focus the probing IR light on a plurality of plasmonic nanostructures 9 or sensing element 10 from which a reflection measurement is to be taken.

In an embodiment, the microfluidic device 17 includes a plurality of microfluidic channels 19 to permit multiplexed neurodegenerative disorder protein detection from a single fluid input sample. FIG. 3B shows for example three microfluidic channels 19. The plasmonic device 3 includes a plurality of sensing areas 31 (three for example for the illustrated embodiment of FIG. 3A) each comprising an assembly of plasmonic nanostructures 9. The sensing area 31 includes at least one or a plurality of the sensing elements 10 with each sensing element 10 including a plurality of plasmonic nanostructures 9. Each microfluidic channel 19 is configured to communicate a fluid or liquid sample to the plasmonic device 3 and to respectively one sensing area 31 and to the plasmonic nanostructures 9 therein. At least a portion of the sensing area 31 defines a portion of the microfluidic channel 19. Each microfluidic channel 19 may include an inlet and an outlet to define a plurality of independent microfluidic channels 19. This permits multiplexed neurodegenerative disorder protein detection to be performed from a single fluid input sample and permits to simultaneously detect different NDD protein biomarkers such as tau and aSyn proteins associated with different diseases.

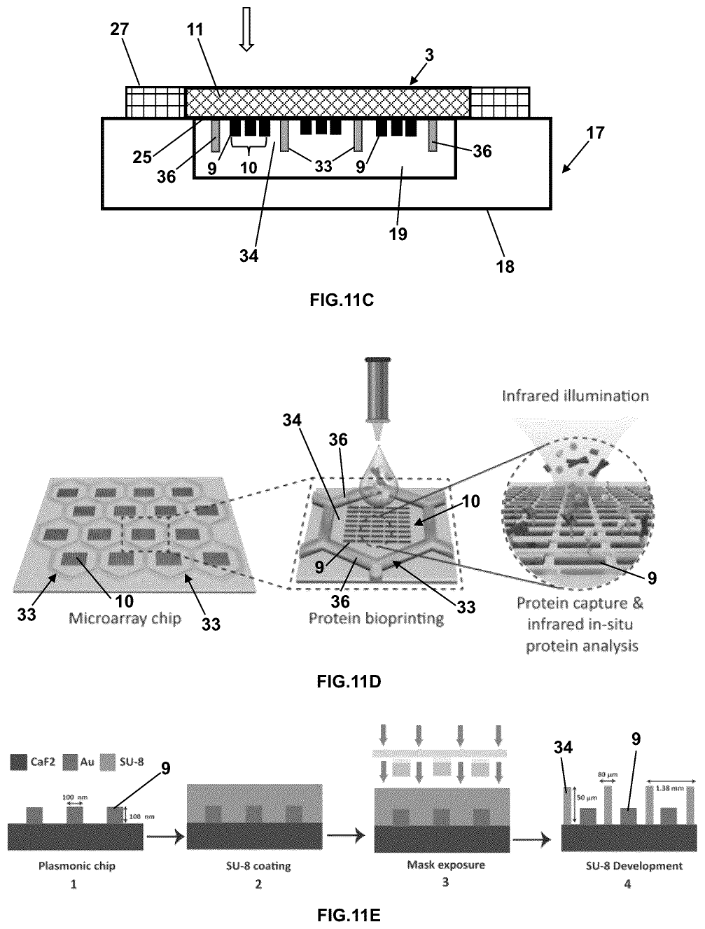

In an embodiment, the plasmonic device 3 includes a plurality of wells or microwells 33 (see for example FIG. 11C). The well 33 includes or defines a chamber or pocket 34 for receiving a sample (for example, a few nanoliters) to be investigated or tested. The sample may for example be provided to the well 33 by bioprinting. The well 33 comprises at least one wall 36 extending from the substrate or support 11. The at least one wall 36 extends to define the chamber or pocket 34 into which the sample is provided. The at least one wall 36 may extend, for example, to define a honeycomb or hexagonal shaped well 33, as shown in the example of FIG. 11D. The plurality of wells 33 may for example form an array of wells 33. Each well 33 may, for example, be interconnected or in contact with at least one other well 33, as shown in the exemplary embodiment of FIG. 11D. Wells 33 may, for example, share at least a portion of the at least one wall 36. Each well 33 is, for example, associated with or includes a plurality of plasmonic nanostructures 9, or at least one sensing element 10 that includes an assembly or plurality of plasmonic nanostructures 9 to capture neurodegenerative disorder proteins of the microwell 33 or that are present inside the well 33. This, for example, enables simultaneous sensing of a plurality of different liquid samples that are provided to different wells 33. The microfluidic channel or chamber 19 of the microfluidic device 17 receives the wells 33 and the plurality of plasmonic nanostructures 9 when the plasmonic device 3 is coupled or attached to the microfluidic device 17, for example, when the support device or chipcell 27 is attached to the flow cell or device 18 so as to position the wells 33 and the plasmonic nanostructures 9 of the plasmonic device 3 in contact with a liquid or fluid present in the microfluidic channel or chamber 19.

For example, 96 wells may be included permitting up to 96 different samples to be tested, in a fast, label-free and high-throughput manner using very small sample volumes, which is highly desired for drug screening assays. This enables simultaneous sensing of same protein conformation incubated with different drugs, or different structural forms with same drug and/or different concentration gradients.

In an exemplary embodiment, the plasmonic device 3 forms, for example, a microarray infrared sensor 3 containing an array of sensing elements 10 composed of, for example periodically arranged, gold nanorods 9 surrounded by SU-8 micro honeycombs or microwells 33.

Each of those combs 33 can then be provided for example bio-printed with a few nLs (for example maximum of 70 nL/well) of a final aggregation assay product containing all/mixture of different structural forms. The proteins are captured on the sensor surface of the sensing elements 10 by, for example, covalently binding to thiols (for example the chip 3 is incubated with N-hydroxysuccinimide-activated carboxyl thiols and spacer OH overnight before spotting by the bio-printer or dispenser).

After bioprinting, the chip 3 is, for example, kept at room temperature for almost 2 hours for incubation. Afterwards, the chip is, for example, washed with PBS (e.g. 1 min) and then water (e.g. 1 min) and dried. Then the chip 3 is placed or maintained in the chipcell 27 and combined with the flowcell 18 with, for example, a buffer such as phosphate buffered saline (PBS) in microfluidic channel or chamber 19 so that measurements can be done with the captured proteins in contact with buffer solution.

Then, the combined microfluidic device (chipcell 27 and flowcell 18) with the microarray chip 3 is kept in the infrared measurement system, for example, an infrared microscope for measurements. The infrared illumination excites the resonance of the nanostructures 9 of the sensing units 10 configured or tuned to overlap with the NDD protein absorption bands. The reference spectra (Ro, without protein) and the protein spectra (R_{NDD}) are measured to retrieve the absorbance signature of protein mixture in that particular well 33 undergoing measurement (−1000*log 10 (R_{NDD}/R₀)). This permits to retrieve the fingerprint absorption signatures of proteins for example in the Amide II band (1500-1600 cm⁻¹) and Amide I band (1600-1700 cm⁻¹) and gives insight into the complex secondary structure information present in the mixture of protein solution.

In-vitro drug screening in neurodegenerative diseases targets, for example, preventing/inhibiting the aggregation of protein biomarkers involved in the causation/progression of NDDs. If the drug works, proteins will remain in monomeric form in majority. If not, they will misfold into different structural forms as time progresses, which results in a mixture of different structural forms at the end. For example, from monomers misfold to oligomers where the alpha-helix/disordered already start changing into beta-sheets, then starts the nucleation of fibril seeds or protofibrils and finally forming mature fibrils, where it is predominantly beta-sheets. The different structural forms can be identified and distinguished by the present invention thus permitting in-vitro drug evaluation. Advantageously, the present invention allows information on the mixed sample with multiple structural forms to be obtained.

FIG. 11E schematically shows an exemplary fabrication process of an exemplary plasmonic device 3 including a plurality of microwells 33. The microwell 33 incudes at least one or a plurality of plasmonic nanostructures or nanoantennas 9. The plasmonic chip 3 is fabricated, as detailed further below, with multiple sensing elements consisting essentially of gold nanostructures 9 using e-beam lithography followed by a development process. Afterwards, the chip 3 proceeds to Photolithography. The plasmonic chip 3 is coated with SU-8 resist to form the microwell structure. The SU-8 resist is exposed with the mask structured with the microcomb/microwell design. The alignment markers on both the chip 3 and the mask help to expose the design of microwells so that they enclose each sensing element 10 within a threshold. Finally, the chip 3 is developed so that exposed regions of SU-8 forms the microwells 33.

More specifically, the plasmonic chip 3 was fabricated on a 3 cm (diameter) CaF₂ substrate consisting multiple arrays of gold nanorods 9 using e-beam lithography. Afterwards, the chip 3 is dehydrated for better adhesion of photolithography resist by Oxygen plasma cleaning. The chip 3 is coated with double layer of SU-8 (5 μm of Kayaku SU8-3005 followed by 50 μm of Kayaku SU8-3050). After soft baking for 20 minutes, the chip 3 is ready for exposure. The mask for SU-8 exposure was fabricated via Direct Laser Writing (VPG200, Heidelberg Instruments) on a 5″ Quartz plate coated with positive resist AZ1512 (500 nm) followed by development, etching and resist stripping in the Hamatech Mask Processor, revealing the microwell design. The chip 3 is exposed then with this mask in top side alignment configuration to transfer the microwell pattern on to SU-8 (Gen3 Mask aligner, i-line). After the Post exposure bake, the chip 3 is developed with PGMEA and IPA to remove the unexposed SU-8 revealing the microwell patterns on the chip 3.

Individual wells 33 can be probed by the IR light, for example, by light focusing via an objective lens 35 on an individual well 33, and using an aperture to spatially limit the light collected by the objective lens 35. The microfluidic device 17 can be displaced relative to the objective lens 35, by for example, a translational stage, to probe and measure each well 33.

Sample volumes may be provided directly to the wells 33 using a dispenser and the plasmonic device 3 attached to the microfluidic device 17 as previously explained in relation to the previous embodiment. Alternatively, the microfluidic device 17 may additionally include one or more the microfluidic channels 19 in fluid communication with the wells 33 to provide sample volumes to the wells 33.

The light source 7 is configured to provide or emit light in a spectrum range corresponding to an absorption spectrum range in which the NDD proteins absorb light and in which light absorption is enhanced by the plasmonic nanostructures 9, for example in the Amide I spectral range. The light source is preferably an infrared light source. The light source may be for example a Globar IR light source such as that commercially available from THORLABS, or for example a tunable quantum cascade laser such a MIRcat laser commercially available from LEONARDO DRS, San Diego.

The system 1 includes the objective lens 35, for example a reflective Cassegrain objective 35, that is arranged to receive light from the light source 7 to direct or focus the incident light onto the plurality of plasmonic nanostructures 9 of the plasmonic device 3 that is positioned in the microfluidic device 17. The focus spot of the objective lens 35 permits to select and determine the plurality of plasmonic nanostructures 9 or the sensing area or element 10 of the plasmonic device 3 that is to be probed or from which a measurement is to be obtained. The system 1 may include a polarizer through which the light provided by the light source 7 passes so as to provide incident light to plasmonic nanostructures 9 that has a polarization parallel to an elongated axis of extension the plasmonic nanostructures 9. An aperture such as a knife-edge aperture may be included, for example, between the objective lens 35 and the plasmonic device 3 to limit the reflected light collected by the objective lens 35 to that reflected from the plurality of plasmonic nanostructures 9 of interest and to block unwanted background light reflected from other parts of the plasmonic device 3. The objective lens 35 may, for example, be part of a microscope which is coupled to the optical detector 5.

The objective lens 35 collects the reflected light and directs the reflected light towards the optical detector 5. The system 1 may include a reflecting optical element 37, for example, a partially reflecting IR mirror 37 that is located between the objective lens 35 and the light source 7 to direct the reflected light provided by the objective lens 35 to the optical detector 5.

The optical detector 5 is configured to receive or detect the reflected light, and to provide measured or detected reflected light spectra. The optical detector 5 may comprise or consist of a photodetector and a spectrometer such as an IR photodetector and spectrometer. The optical detector 5 may comprise or consist of at least one spectrometer, for example, a Fourier-transform infrared FTIR spectrometer, and/or a detector or point detector such as a liquid nitride cooled mercury cadmium telluride detector. The optical detector 5 may, for example, comprise or consist of at least one imaging IR detector, such as a pixelated IR camera. The optical detector 5 is configured to determine reflected light spectra and to provide or communicate the reflected light spectra for further analysis, for example, as a reflected light spectrum electronic signal or reflected light spectrum data.

The NDD biosensing system 1 may also include at least one data processing device 39. The data processing device 39 operatively connected to the optical detector 5 to receive the reflected light spectrum data or signal.

The data processing device 39 may include at least one processor 41 and may include at least one storage or memory device 43. The data processing device 39 processes information that may be transmitted from a plurality of electrical and electronic devices that include, without limitation, the optical detector 5 to which the data processing device 39 is operatively coupled for communication therebetween, or in operative connection thereto. The data processing device 39 may, for example, be in operative connection with a display device for displaying calculation or analysis results and include a graphics processor configured to perform image generation. The data processing device 39 may, for example, be configured to operate and display a graphical user interface. The data processing device 39 may, for example, be configured to communicate calculation or analysis results to external devices with which the data processing device 39 is operatively coupled for communication and/or in operative connection.

Intra-device communication, such as communication between the data processing device 39 and the detector 5, may for example be via a wired and/or a wireless communication technology. Each device includes a communications module or communication circuitry configured to communicate data between the devices. Wireless communication may, for example, be via near field communication technology such as WiFi or Bluetooth. Wired communication may for example be used such as a Local Area Network (LAN) using for example an ethernet communications protocol.

In a non-limiting exemplary embodiment, the data processing device 39 may for example comprise a computer such as a desktop or laptop computer, a tablet, or a smartphone.

The processor 41 may comprise one or more processors or microprocessors or processing devices, or microcontrollers, microcomputers, programmable logic controllers (PLC), or application specific integrated circuits, and other programmable circuits.

Each “processor” herein includes processing circuitry, and/or may include multiple processors. For example, as used herein, including the claims, the term “processor” may include various processing circuitry, including at least one processor, wherein one or more of at least one processor, individually and/or collectively in a distributed manner, may be configured to perform various functions described herein. As used herein, when “a processor”, “at least one processor”, and “one or more processors” are described as being configured to perform numerous functions, these terms cover situations, for example and without limitation, in which one processor performs some of recited functions and another processor(s) performs other of recited functions, and also situations in which a single processor may perform all recited functions. Additionally, the at least one processor may include a combination of processors performing various of the recited/disclosed functions, e.g., in a distributed manner. At least one processor may execute program instructions to achieve or perform various functions.

The storage or memory device 43 may, for example, comprise a computer-readable medium or computer-readable memory for example a semiconductor memory, or a non-volatile medium or memory such as flash memory; or comprise for example a hard disk drive HDD. The storage or memory device 43 may be removable or non-removable. The computer-readable medium or memory is, for example, a non-transitory computer-readable medium or memory.

The storage or memory device 43 stores and transfers information and instructions to be executed by processing device 39. The memory or storage device 43 can also be used to store and provide temporary variables, static information and instructions, or other intermediate information to the data processing device 39 during execution of instructions by the data processing device 39.

Instructions that are executed include, but are not limited to, instructions for analysis of signals and of data transmitted from the optical detector 5. The execution of sequences of instructions is not limited to any specific combination of hardware circuitry and software instructions.

Instructions may, for example, include instructions or computer instructions executable on the processor or data processor 41. Instructions may, for example, be in the form of processor or computer instruction code.

The optical detector 5 may optionally also include a data storage device coupled to a processing device of the optical detector 5. The data storage device may, for example, temporarily store the data generated from the measured optical signal detected by the optical detector 5.

The storage or memory device 43 includes, for example, one or more computer programs 45 comprising instructions permitting to manage and operate the calculations relating to the reflected optical spectra and the analysis of the reflected optical spectra mentioned herein. For example, custom-made Matlab functions may be used for such calculations and analysis.

The methods, spectral calculation and spectral analysis described herein may be encoded as executable instructions embodied in a computer readable medium, including, without limitation, the storage device or memory device 43. Such instructions, when executed by a processing device 39, cause the processing device 39 to perform at least a portion of the methods, spectral calculation and spectral analysis described herein.

The data processing device 39 is configured to process the reflected light spectrum data or signal to determine or calculate an absorption spectrum representing absorption by NDD proteins captured by the functionalized plurality of plasmonic nanostructures 9 from which the reflected light was measured by the system 1.

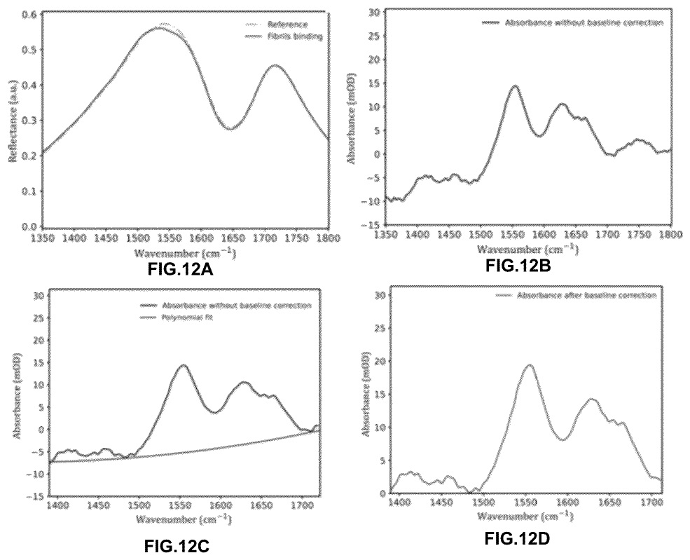

The absorption spectrum can be calculated in a manner classically known based on a reference reflected light spectrum measured or determined prior to capture of any NDD proteins, that is, a reflected light spectrum measured from the functionalized plurality of plasmonic nanostructures 9 in a fluid or liquid prior to the provision of a sample-to-be-investigated being introduced to the microfluidic device 17. The reference reflected light spectrum data or signal is also provided or communicated to the data processing device 39. For example, the absorption spectrum Abs representing absorption by captured NDD proteins can be calculated or determined by the data processing device 39 according to Abs=Blog₁₀ (R_{NDD}/R₀), where ABS is the differential absorbance, B is a constant, R_{NDD} is the measured reflected light spectrum measured or determined after NDD protein capture or binding, and R₀ is the reference reflected light spectrum measured or determined prior to capture or binding of any NDD proteins. A baseline correction may also then be applied, where a determined baseline is subtracted from the determined differential absorbance ABS to provide a baseline-corrected absorbance spectrum (see, for example, FIGS. 12A to 12D).

The data processing device 39 is configured to determine or calculate a plurality of absorption spectra determined from the provided or communicated reflected optical infra-red spectra. The plurality of absorption spectra preferably comprises time-resolved absorption spectra, that is, chronological absorption spectra or absorption spectra arranged in the order of time. In particular, the absorption spectra may represent time-resolved infra-red absorption by neurodegenerative disorder monomeric proteins and/or protein secondary structure types formed from neurodegenerative disorder aggregated proteins, for example, first protein secondary structure types 49 formed from neurodegenerative disorder aggregated proteins and second protein secondary structure types 51 formed from neurodegenerative disorder aggregated proteins (see, for example, FIG. 2A), when such entities or proteins are present in the sample under investigation and are captured by the functionalized plasmonic nanostructures 9. Distinct structural species are differentiated using their unique absorption signatures.

Advantageously, the data processing device 39 is configured to process the plurality of absorption spectrum signals to identify the first protein secondary structure type 49 and the second protein secondary structure type 51, and to distinguish the identified first protein secondary structure type 49 from the second protein secondary structure type 51, and the identified second protein secondary structure type 51 from the first protein secondary structure type 49.

In an embodiment, the data processing device 39 is configured to process the plurality of absorption spectrum signals to identify the neurodegenerative disorder monomeric protein 47, the first protein secondary structure type 49 and second protein secondary structure type 51, and to distinguish the identified first protein secondary structure type 49 from the second protein secondary structure type 51, the identified second protein secondary structure type 51 from the first protein secondary structure type 49 and the identified neurodegenerative disorder monomeric protein 47 from the first and second protein secondary structure types 49, 51.

The first protein secondary structure type 49 is different to the second protein secondary structure type 51 and to the neurodegenerative disorder monomeric protein 47. The neurodegenerative disorder monomeric protein 47 and the first and second protein secondary structure types 49, 51 preferably are evolving and distinct intermediate structural species of a neurodegenerative disorder protein-aggregation body formation process. For example, these may be different structural species such as monomers, oligomers, and fibrils present on the pathway Lewy body formation.

The data processing device 39 is configured to process the plurality of absorption spectra to determine the simultaneous presence of the first protein secondary structure type 49 and the second protein secondary structure type 51 by the plurality of plasmonic nanostructures 9 of the plasmonic device 3.

In one exemplary embodiment and described in more detail below, the neurodegenerative disorder monomeric protein 47 may include or consists essentially of an alpha-synuclein monomeric protein, the first protein secondary structure type 49 may include or consists essentially of an alpha-synuclein oligomer, and the second protein secondary structure type 51 may include or consist essentially of an alpha-synuclein fibril.

The data processing device 39 is configured to process the plurality of absorption spectra to determine distinguishing contributions to the absorption spectra by a secondary structure of the monomeric protein or by a secondary structure of the first or second protein secondary structure types formed from aggregated proteins. The data processing device is configured to distinguish the first protein secondary structure type 49 from the second protein secondary structure type 51, the second protein secondary structure type 51 from the first protein secondary structure type 49 and the neurodegenerative disorder monomeric protein 47 from the first and second protein secondary structure types 49, 51 by determining a relative absorption contribution of a plurality of different constituent secondary structure structural motifs 53 (see, for example, FIG. 2C). In one exemplary embodiment, the constituent secondary structure structural motifs include at least one of: alpha helices, beta-sheets and beta-turns. The data processing device 39 is configured to process the plurality of absorption spectra to determine a second derivative of an absorption value over a spectral range of the absorption spectra, or a rate of change of [a rate of change of an absorption value over a spectral range of the absorption spectra]. This allows to identify peak positions of individual contributing bands of different structural motifs, which are then used as a reference to deconvolute absorption contributions by a plurality of different constituent secondary structure structural motifs. Deconvolution may be performed using, for example, Fourier Self-Deconvolution (see for example, Jyrki K. Kauppinen, Douglas J. Moffatt, Henry H. Mantsch, and David G. Cameron, “Fourier Self-Deconvolution: A Method for Resolving Intrinsically Overlapped Bands,” Appl. Spectrosc. 35, 271-276 (1981), the disclosure of which is hereby incorporated herein by reference in its entirety for all purposes). This conformational profiling allows to discriminate the different structural species, as detailed further below.

The system 1 forms an immunoassay coupled optofluidic surface-enhanced IR absorption (ImmunoSEIRA) sensor or device that detects proteins linked to NDDs with specificity and differentiates the distinct structural species using their unique absorption signatures. The system 1 is a structural biomarker sensor.

In an embodiment, the data processing device 39 is configured to process an absorption spectrum to determine a quantity ratio or concentration ratio of (i) a first protein secondary structure type to (ii) a second protein secondary structure type. The data processing device 39, for example, the storage device or memory device 43, includes a trained deep neural network configured to process an absorption spectrum or absorption spectra to determine a first protein secondary structure type to second protein secondary structure type quantity ratio or concentration ratio when the absorption spectrum is inputted to the trained deep neural network. The inputted absorption spectrum may be an absorption spectrum determined from the reflected optical spectrum provided by the optical device 5, or may be provided or communicated to the data processing device 39 by a device external to the system 1 that is requesting the data processing device 39 to provide a quantity ratio or concentration ratio. For example, the first protein secondary structure type to second protein secondary structure type quantity ratio or concentration ratio is an oligomer to Fibril quantity ratio. Further details of training the deep neural network to obtain the trained deep neural network and the resulting predictive accuracy of the trained deep neural network are set out below.

NDD biosensing is performed by providing a fluidic sample to the plasmonic device 3, the plurality of plasmonic nanostructures 9 being functionalized and having attached thereto capturing agents 15 configured to bind to neurodegenerative disorder monomeric proteins 47 and to protein secondary structure types 49, 51 formed from neurodegenerative disorder aggregated proteins. Reflected infrared spectra, reflected from the plasmonic device 3, are detected and obtained. Absorption spectra are determined from the obtained reflected optical infra-red spectra. The absorption spectra represent time-resolved infra-red absorption by at least one of: (i) neurodegenerative disorder monomeric proteins 47, (ii) first protein secondary structure types 49 formed from neurodegenerative disorder aggregated proteins and (iii) second protein secondary structure types 51 formed from neurodegenerative disorder aggregated proteins.

From the plurality of absorption spectra, at least one of the first protein secondary structure type 49 and the second protein secondary structure type 51 is identified, and the identified first protein secondary structure type 49 is distinguished from the second protein secondary structure type 51, and/or the identified second protein secondary structure type 51 is distinguished from the first protein secondary structure type 49. As a result, the NDD biosensing method allows to detect, identify, and distinguish different protein secondary structure types that are misfolded protein structural biomarkers associated with NDDs from the contribution that these structural biomarkers make to IR absorption spectra.

From the plurality of absorption spectra, at least one of the neurodegenerative disorder monomeric protein 47, the first protein secondary structure type 49 and the second protein secondary structure type 51 can be identified, and the identified first protein secondary structure type 49 is distinguished from the second protein secondary structure type 51, the identified second protein secondary structure type 51 is distinguished from the first protein secondary structure type 49, and the identified neurodegenerative disorder monomeric protein 47 is distinguished from the first and second protein secondary structure types 49, 51.

As previously mentioned, distinguishing can be done, for example, by determining a relative absorption contribution of different constituent secondary structure structural motifs.

A first protein secondary structure type to second protein secondary structure type quantity ratio may be determined by inputting an absorption spectrum to a trained deep neural network trained to determine a first protein secondary structure type to second protein secondary structure type quantity ratio.

While in one exemplary embodiment, the first protein secondary structure type 49 may include or consists essentially of an alpha-synuclein oligomer, and the second protein secondary structure type 51 may include or consist essentially of an alpha-synuclein fibril, further protein secondary structure types, such as third and fourth different protein secondary structure types, that are present can be identified and distinguished in the same manner.

Protein misfolding has been identified as a key event in NDD disease progression. It is considered that healthy proteins misfold into abnormal forms implicated in the development and progression of NDDs, first into oligomers in early stages and into fibrils in later stages of the disease. That is, there is a change in protein structure. The system and method of the present invention allows to identify and distinguish these specific misfolded abnormal forms each having different protein structure via their different protein secondary structure type. In particular, the system and method differentiate the distinct structural species using their unique absorption signatures thus permitting to detect tell-tale signs of a NDD disease by assuring an accurate determination of the presence of these different protein aggregates. This permits for early detection and monitoring of NDDs, and can be used for assessing treatment options at various stages of the disease's progression.

An embodiment of the present invention also concerns a non-transitory computer readable medium having computer code recorded thereon, the computer code configured to perform a neurodegenerative disorder biosensing method when executed on a data processing device of a computer device, the method comprising:
processing a plurality of absorption spectra determined from reflected optical infra-red spectra, the plurality of absorption spectra representing time-resolved infra-red absorption by at least one of: (i) first protein secondary structure types 49 formed from neurodegenerative disorder aggregated proteins and (ii) second protein secondary structure types 51 formed from neurodegenerative disorder aggregated proteins;
processing the plurality of absorption spectra to identify at least one of the first protein secondary structure type (49) and the second protein secondary structure type 51 and to distinguish the identified first protein secondary structure type 49 from the second protein secondary structure type 51 and the identified second protein secondary structure type 51 from the first protein secondary structure type 49.

The plurality of absorption spectra may represent time-resolved infra-red absorption by at least one of: (i) neurodegenerative disorder monomeric proteins 47, (ii) first protein secondary structure types 49 formed from neurodegenerative disorder aggregated proteins and (iii) second protein secondary structure types 51 formed from neurodegenerative disorder aggregated proteins. Processing of the plurality of absorption spectra is performed to identify at least one of the neurodegenerative disorder monomeric protein 47, the first protein secondary structure type 49 and the second protein secondary structure type 51, and to distinguish the identified first protein secondary structure type 49 from the second protein secondary structure type 51, the identified second protein secondary structure type 51 from the first protein secondary structure type 49 and the identified neurodegenerative disorder monomeric protein 47 from the first and second protein secondary structure types 49, 51.

The system 1 and NDD biosensing method permit to detect proteins linked to NDDs with specificity and differentiate the distinct structural species using their unique absorption signatures. The system 1 can retrieve time-resolved absorbance fingerprints and is capable of multiplexing for the simultaneous monitoring of multiple pathology-associated biomarkers. The system 1 allows diagnosis of NDDs, disease monitoring, and evaluation of novel therapies. Further details of exemplary embodiments and results achieved by the system and method of the present disclosure are now provided below.

In an exemplary embodiment, the system 1 essentially forms an immunoassay coupled optofluidic Surface-enhanced IR absorption (SEIRA) sensor 1 capable of extracting unique structural fingerprints of different conformational species of one of the NDD biomarkers, that is for example alpha-synuclein aSyn, including its oligomeric and fibrillary aggregates. The sensor chip 3 exploits a nanoplasmonic metasurface including for example engineered gold nanorod arrays 9 that are functionalized with antibodies 15 for specific protein detection. The chip 3 is fabricated in a two-dimensional (2D) microarray format and integrated with microfluidics 17 to facilitate in situ capture and structural analysis of target protein 47, 49, 51 in minute sample volumes. The near-field enhanced amide II and I absorption signals of the ImmunoSEIRA sensor 1 can optionally be aided by artificial intelligence (AI) to quantitatively identify the percentage presence of, for example, oligomers and fibrils such as aSyn oligomers and fibrils in their mixture. The different sensing elements 10 of the microarray chip 3 can, for example, be selectively functionalized with two different types of antibodies 15, for example, specific to the tau protein and aSyn for multiplexed biomarker detection using spectroscopic signals. Results presented herein show this structural biosensor 1 to be able to retrieve pathological protein biomarker absorption signatures from the complex biomatrix of human CSF bringing one step closer, the use SEIRA sensors 1 for clinical-based diagnostics of NDDs.

Optofluidic plasmonic SEIRA is realized in the structural biomarker sensor 1. FIG. 2A shows different exemplary structural aSyn species such as monomers, oligomers, and fibrils present on the pathway Lewy body formation. FIG. 2A to 2C presents an overview of an exemplary measurement system and method of the present disclosure as well as presenting the detection principle of plasmonic SEIRA as a structural biomarker sensor 1.

In the exemplary embodiment, the optofluidic sensor 1 includes the plasmonic chip 3 placed in a polydimethylsiloxane (PDMS) part 17 incorporating microfluidic channels 19. Both the IR illumination and the reflected signal are collected from the backside of the chip 3 in plasmonic internal reflection (PIR) configuration to extract protein secondary structure signatures despite the overlap from aqueous buffer absorbance (FIG. 2B). In this embodiment, SEIRA is combined with immunoassay (ImmunoSEIRA) to selectively capture aSyn species for structural-based differentiation (FIG. 2B). The plasmonically enhanced absorption signatures from proteins in amide II and amide I bands (1500 to 1700 cm⁻¹) are retrieved by spectral reflection sensing (FIG. 2C). The amide I band (1600 to 1700 cm⁻¹) absorption contributed by C═O and C—N vibrational modes envelopes the contributions from individual secondary structure motifs that are variably present in different conformational states of the same protein, i.e., the disordered and a helix motifs have their structural fingerprints around 1643 to 1660 cm⁻¹, B turns absorb around 1667 to 1685 cm⁻¹, whereas the main component of the aggregate species, B sheets, has its absorption fingerprint in the lower wave numbers of 1615 to 1635 cm⁻¹ as well as in the higher wave numbers between 1688 and 1696 cm 1 (FIG. 2C). This enables to differentiate aSyn monomers from the pathological species of oligomers and fibrils with each having their own distinct spectral contributions that can be mapped using the system and method of the present invention.

In an exemplary embodiment, the nanoplasmonic structures 9 of the chip 3 were fabricated on an IR transparent calcium fluoride (CaF2) substrate in a microarray format comprising three parallel rows, where each row contains linearly arranged metasurface elements 9 and gold mirrors 12 for sensing and referencing, respectively (FIG. 3A). Each of these metasurface sensing elements 10 is formed of periodically arranged unit cells consisting of plasmonic nanorods 9 coupled with grating order and nanogaps (see for example, A. John-Herpin, A. Tittl, H. Altug, Quantifying the limits of detection of surface-enhanced infrared spectroscopy with grating order-coupled nanogap antennas. ACS Photonics 5, 4117-4124 (2018), which is hereby incorporated herein by reference in its entirety for all purposes). The dimensions of the nanorod length L, the gap G between the rods in the x direction, and the y-axis periodicity P_{y} are in this exemplary embodiment L=1.5 μm, G=0.08 μm, and Py=3.2 μm, respectively (FIG. 3A right).

The sensor chip 3 is then placed in a PDMS part (chipcell 27; FIG. 3B, left). A microfluidic flowcell 18 is custom-made to continuously run the buffer and inject samples over the sensor 3, including three independent channels 19 with an exemplary width of 300 μm, depth of 30 μm, and length of 3.5 mm (micro-flowcell 18; FIG. 3B right). The designed micro-flowcell 18 with less than 35-nl channel volume helps to reduce the amount of required sample and amplifies the antibody-antigen interaction, thereby leading to higher binding performance. These independent channels 19 are positioned such that when combined with the chipcell 27, the three channels 19 overlap with the three respective rows of the sensing elements 10 and mirrors 12.

The parameters of the nanorod 9 arrays are optimized to overlap the plasmonic resonance with the amide II and amide I bands of the protein fingerprints, i.e., between 1500 and 1700 cm⁻¹, with the peak around 1600 cm⁻¹ in aqueous conditions. Such engineered nanostructures 9 provide high sensitivity by enhancing the absorption signals from small quantities of protein on the surface compared to conventional IR spectroscopy methods, thereby opening up immense possibilities for IR-based diagnostic applications.

The design parameters are first evaluated using simulation based on a frequency domain solver, and then the optical response is validated through measurement with the optofluidic sensor 1 (FIG. 3C). Details on the fabrication and design of the nanostructure and microfluidic parts are detailed further below. In parallel, the generation and characterization of aSyn species are performed using well-established and optimized protocols (see for example reference 53). FIG. 3D shows the transmission electron microscope (TEM) images of the three distinct aSyn species-monomers, oligomers, and fibrils. Monomers are disordered and smaller in dimensions, so they are not visible in the TEM grid (top). As reported previously, the unmodified oligomers exist as a mixture of different morphologies, such as spherical, tubular, and ring-like structures of different sizes (middle) and are characterized by mixed secondary structures. The fibril samples show fragmented filamentous structures, which are rich in cross β sheet structures, with an average length and width of 50 to 200 nm and 5 to 20 nm, respectively (bottom).

The spectral specificity of the sensor 1 provides an inherent advantage for a structural biomarker-based diagnostic approach. Target selectivity becomes crucial in the clinical setting where body fluids contain numerous biomolecules. Hence, the strengths of immunoassay and IR spectroscopy together are leveraged on the SEIRA sensor 1 to capture aSyn species using well-characterized and validated aSyn-specific antibodies 15.

The workflow of the immunoassay is a simple three-step procedure, as shown in FIG. 4A. The first step is thiolation. The plasmonic sensor chip 3 is incubated in a mixture of N-hydroxysuccinimide (NHS)-activated carboxyl thiols and OH spacer thiols to form a uniformly spaced monolayer of the activated NHS esters on the plasmonic surface. This facilitates the covalent coupling of any molecules with free amino groups to attach to the surface. After overnight incubation, the chip 3 is washed and mounted with the microfluidic parts of the optofluidic system 17 to commence the continuous in situ spectroscopic measurement. Flowing of running buffer [phosphate-buffered saline (PBS, 1×)] through the middle fluidic channel 19 of the micro-flowcell component 17 (FIG. 3A, middle box) continuously is first carried out. Light is focused on one of the sensing elements 10 of the sensing area 31 while measuring a gold mirror 12 intermittently as a reference.

From these continuous reflectance spectra, time-resolved absorbance signals are extracted over the amide II and I bands (1500 to 1700 cm⁻¹) by normalizing the measured spectrum at each time point to the baseline established during the thiolation step. The details of the spectral data acquisition are provided further below. The baseline correction procedure performed to retrieve the absorbance signals shown in the main text figures is explained with reference to FIGS. 12A to 12D.

FIG. 12A shows the plasmonic resonance of the sensing element 10 tuned to resonate around 1600 cm⁻¹ in wet conditions (‘Reference’, R₀) and the resonance spectrum collected after the aSyn fibrils have bound to the thiols 15 on the sensor surface (‘Fibrils binding’, R_{NDD}). The spectral shift in FIG. 12A, as well as the dips in the reflection spectrum of the latter, indicates the successful binding of aSyn fibrils on the surface. FIG. 12B shows the differential absorbance (mOD) of the aSyn fibrils calculated as −1000*log 10 (R_{NDD}/R₀), and the obtained result is shown. The asymmetric line profile obtained for the protein absorption bands is clearly visible. As seen in FIG. 12C, one can then perform a baseline correction to get a final absorption spectra, which are shown as the results in the figures in the main text and the supplementary information. The obtained absorption spectra was fitted with a second-order polynomial (lower curve). FIG. 12D shows, after subtracting the polynomial from the spectra, the retrieved baseline-corrected absorbance spectra, which show vibrational features as known from conventional IR spectroscopy.

For monitoring target binding on the surface and the interaction kinetics, time-resolved absorbance signals are integrated over the amide range (1525 to 1650 cm⁻¹) to obtain the sensorgram, as shown in FIG. 4B.

The measurement of the thiolated sensor 3 in the buffer is continued for ˜30 min to get a smooth and uniform baseline. In this time window, the time-resolved absorbance is zero as there is no change relative to the defined baseline (FIG. 4C, thiolated sensor). Next, one proceeds to the second step-antibody immobilization. Here, a sequence-specific antibody targeting the flexible C-terminal domain of the protein: SYN-211 is chosen, as the capturing agent 15 that binds to full-length aSyn irrespective of its conformation, thereby enabling unbiased structural analysis of the protein. The antibody solution is injected at ˜30 min, and one notes an increase in the sensorgram, indicating the binding of the antibody on the thiolated sensing element 10 (FIG. 4B shaded region). Once the antibody solution completely flows over the surface, the running buffer is introduced back into the channels at ˜75 min. The unbounded antibody is washed away as indicated by a small dip at the beginning of the buffer wash, and then one can observe a permanent increase in the sensorgram signal even after washing (˜75 to 110 min). This indicates the immobilization of a sturdy capture layer of antibodies 15. The time-resolved absorbance spectra of this second step (between ˜30 and 110 min) retrieve the amide II and I bands of the antibody (FIG. 4C, antibody immobilization). After one obtains a steady signal for the antibody step, one progresses to the final step of this immunoassay-aSyn capture. For demonstration in the Figure, pure species of aSyn fibrils are used. Two hundred microliters of this target protein solution is injected at around ˜110 min at a concentration ensuring protein enrichment on the sensor surface. The increase in the sensorgram signal (FIG. 4B right shaded region) indicates the enrichment of aSyn fibrils on the sensor surface. After the course of the protein solution, the buffer is reintroduced to the channel for washing at ˜140 min. The steady sensorgram signal (between ˜140 and 250 min) conveys the successful capture and retention of the aSyn fibrils by the antibody layer even after the washing step. The time-resolved absorption spectra of this final step reveal the absorption signatures of aSyn fibrils from injection to stable binding (FIG. 4C aSyn fibrils capture). One can clearly see its expected distinct spectral shape in the amide I region (1600 to 1700 cm⁻¹) with a peak around ˜1630 cm⁻¹ and an off-shoulder around higher wave numbers, indicative of high β sheet content. The binding kinetics of aSyn oligomers and monomers and their respective absorbance spectra are provided in FIGS. 4D and 4E, respectively. FIG. 4F shows the results of the control experiment with and without a blocking step in the functionalization assay for the in-flow experiments using pure aSyn fibrils form.

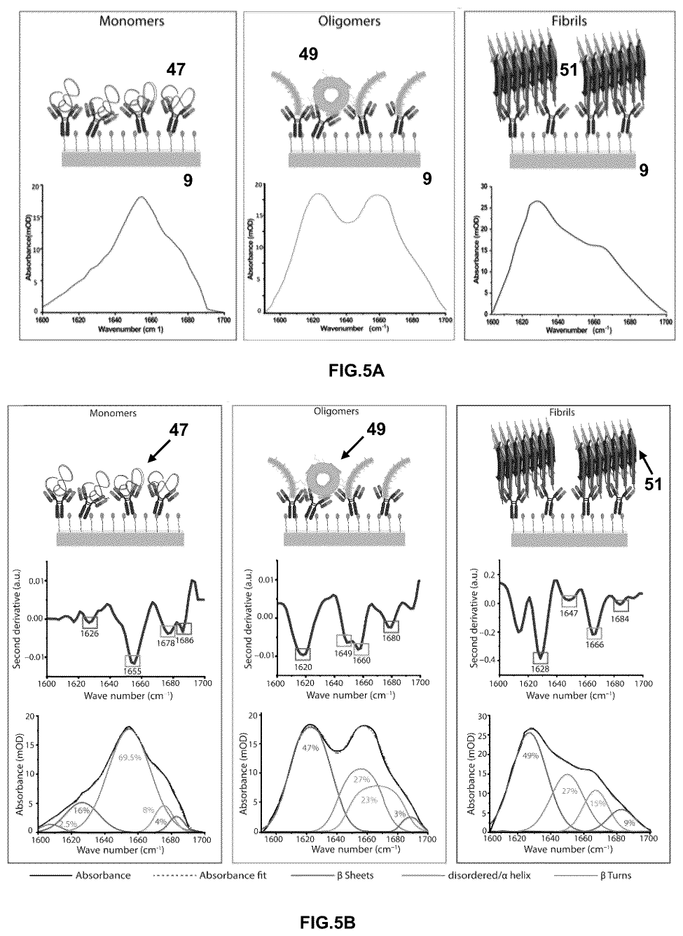

To show conformational profiling discriminating the different structural species of the aSyn, the ability of our sensor 1 to identify the secondary structure of the three aSyn species, i.e., monomers 47, oligomers 49, and fibrils 51, by conducting an in-depth analysis of their amide I band for conformational profiling was investigated. This is done independently using, for example, two conventional and well-established mathematical methods: second-derivative analysis and Fourier self-deconvolution (FSD) with curve fitting that helps to deconvolute the substantially overlapped component bands arising from multiple secondary structural motifs (see for example references 39 and 56). Using unbiased second-derivative analysis, one identifies the peak positions of the individual contributing bands of different structural motifs. Using these peak positions as a reference, one then executes and optimizes the FSD to extract quantitative data of the individual band contributions. FIG. 5B shows the complete analysis of aSyn monomers 47 (left), oligomers 49 (middle), and fibrils 51 (right). Preferably, one specifically focuses on three main structural bands-disordered/a helix, β sheets, and β turns. For clarity, the original absorbance spectra of the monomers, oligomers, and fibrils used in this deconvolution analysis are shown in FIG. 5A.

As seen in the lower graphs of FIG. 5B, aSyn monomers 47 have a larger amount of disordered structures with almost 70% contribution and only 20% of β sheet outlining its native unstructured form. In fibrils 51, the dominant structure with almost 58% contribution is β sheets, which is acknowledged to be a characteristic of the fibrillar pathological species. The disordered content in fibrils is only 27%, and the rest is contributed by β turns that help to connect and form antiparallel β sheets. In the heterogenous population of unmodified oligomers 49, it is found mostly the presence of β sheet with a 50% contribution and the same contribution of disordered structure as in fibrils (27%) but with the increased presence of β turns (23%). Using distinct spectroscopic absorption signals, structural differences are identified between all three species 47, 49, 51, despite the conformational similarities of oligomers 49 and fibrils 51. Notably, the present invention makes it possible to decipher the structural components of oligomer species 47 (FIG. 5B), thus allowing for the use of aSyn species-specific structure-based biomarkers in an immunoassay-based diagnostic tool, the system 1 of the present invention embodying such a tool. This capacity to distinguish oligomers 49 and fibrils 51 is important as the oligomeric forms of aSyn have been implicated to play important roles in aSyn toxicity and pathology spreading during disease progression. Since the present method relies on the attachment of proteins on the surface by functionalization, it was verified and confirmed that this does not alter the native conformations of immobilized proteins.

In an embodiment, AI-aided ImmunoSEIRA is implemented to quantitatively predict the percentage of aSyn oligomers and fibrils in a mixture. In a patient sample, it is likely that the three forms of aSyn species are present at different levels simultaneously. Although there are aSyn antibodies that bind preferentially to aggregated forms of aSyn, there are no antibodies that can selectively capture either oligomers or fibrils. This has hampered efforts to accurately quantify these aggregate species and/or correlate their levels to disease stages, symptoms, or rate of progression. Therefore, the present invention provides a method that enables accurate quantification of each form of aSyn, including oligomers 49 and fibrils 51. This also enables accurate assessment of total aSyn in biological samples.

The spectral decomposition approaches used in FIG. 5B cannot quantitatively accurately predict the percentage composition of oligomers 49 and fibrils 51 in a mixture because they only provide the distribution of individual structural motifs (e.g., disordered and β sheet). This information is not sufficient to spectrally regress and associate these subbands quantitatively to different aggregates to reconstruct their percentage distribution. This is addressed by combining AI with spectral analysis using ImmunoSEIRA to accentuate the unique absorption signatures of oligomers 49 and fibrils 51 rather than their underlying individual secondary structure motifs. Machine learning and AI analysis is combined with IR spectroscopy to address this inconvenience and more particularly AI is combined with SEIRA to discriminate among the different aggregation states of the same protein when present in a mixture.

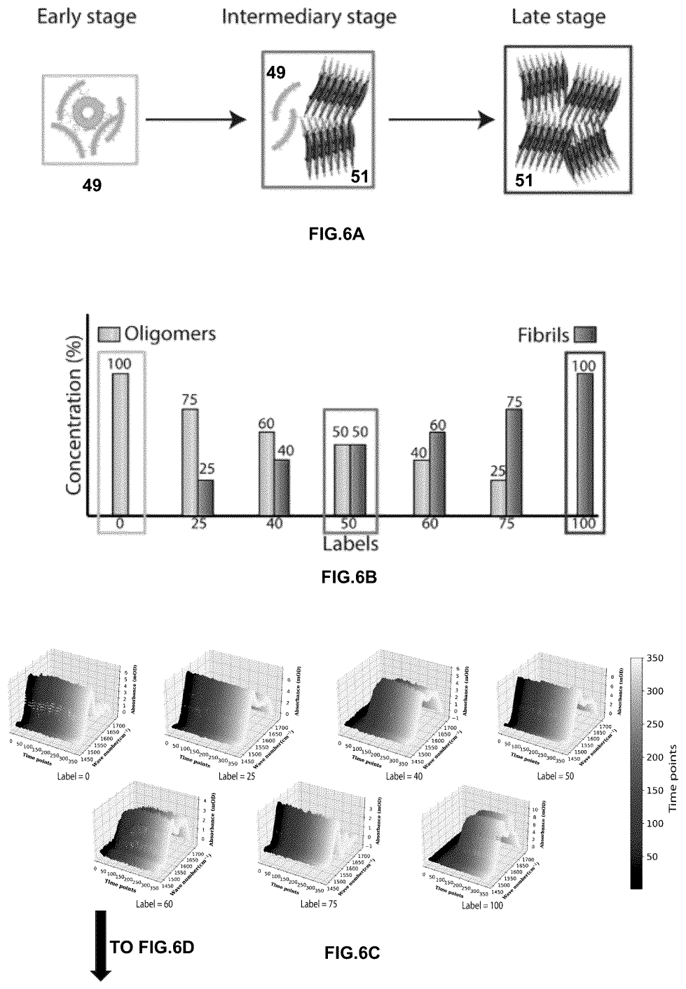

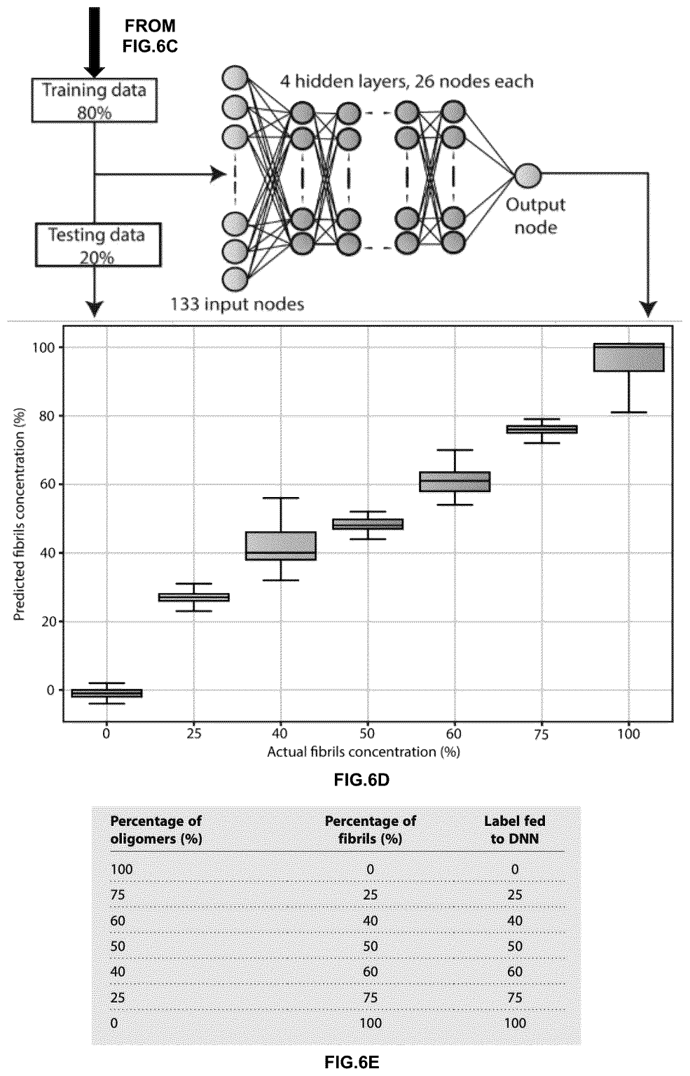

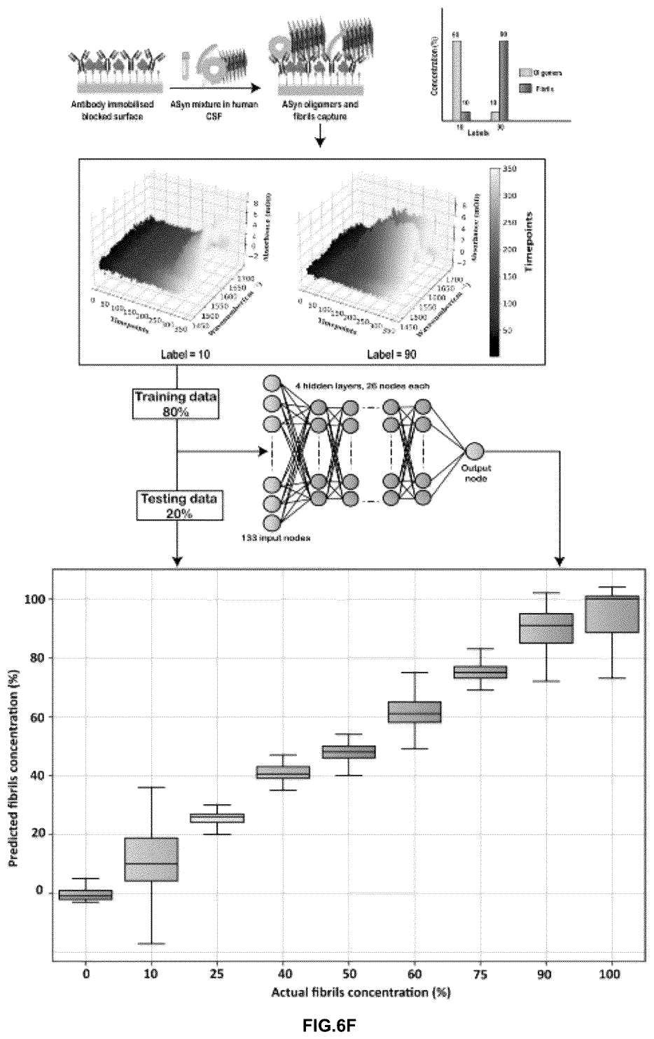

To determine whether a combined AI-ImmunoSEIRA approach could distinguish quantities of oligomers 49 from fibrils 51, a plausible scheme of total aggregate presence in a sample at different stages of the disease is followed, with mostly oligomers 49 in the beginning but with the presence of fibrils 51 gradually increasing and eventually becoming the dominant species in the mixture (FIG. 6A). Based on this, a sample set of different mixtures of titrated percentage combinations of aSyn oligomers and fibrils: 100-0, 75-25, 60-40, 50-50, 40-60, 25-75, and 0-100% (oligomers-fibrils), respectively were provided (FIG. 6B). 3D spectrograms comprising absorbance-wave number-time points for these different sample sets were measured using the optofluidic ImmunoSEIRA setup or system 1 (FIG. 6C).

The time-resolved spectral analysis generates a large set of data with close to 3.5 million data points, thus providing an ideal opportunity to use AI-DNN (deep neural network) models. One preferably focuses on the spectro-temporal range containing information on the amide II and I absorption bands that vary over time from the gradual binding of the aggregates over the surface up until stabilization. Each of such spectra contains 133 data points that correspond to time-varying absorbance in the wave number range of 1450 to 1700 cm⁻¹ with 4-cm⁻¹ spectral resolution. This data cube was then concatenated with labels assigned to each mixture ratio as 0, 25, 40, 50, 60, 75, and 100 in the order above that correlates to the percentage presence of fibrils (see FIG. 6E) to form the dataset quartet. A DNN was modelled based on a multilayer perceptron (MLP) model and it is applied to the data quartet. The exemplary DNN was configured with 133 input nodes—each node for feeding the absorbance at a given wave number and a given time point and one output node to predict the label of the fed absorbance spectra. The entire dataset was split randomly into two, 80 and 20%. The 80% data quartet of each combination ratio was used for training and validating the neural network for the best possible prediction accuracy by performing cross-validation (CV) and parameter tuning for optimization. This was done with fivefold CV to find the optimum number of hidden layers and nodes that can yield the desired performance in terms of the least mean squared error for prediction. This training step yielded the DNN model consisting of 4 hidden layers with 26 nodes each, with an overall accuracy score of 94.66%.

After this validation, the remaining 20% of the entire dataset of every combination ratio that has not been included in the training was fed to the network to evaluate the prediction accuracy for the regression percentages (i.e., the presence of oligomers and fibrils in percentage). From FIG. 6D, one infers that, in this test, the network predicts the percentages of unknown sample ratio with excellent accuracy, and the average of the predicted percentage of fibrils in the mixtures matches the actual concentration percentage.

Notably, in this DNN analysis, raw spectral data was used, including initial time points where the absorption signal is weak and noisy. Despite the low signal levels, without any smoothing and averaging, the AI-aided ImmunoSEIRA sensor is robust and structurally sensitive. This permits to detect and quantify aSyn oligomers and fibrils and can pave the way to correlate their ratios during disease progression in longitudinal studies where the physiological concentrations are expected to be low. Therefore, the present approach can advantageously enable a more accurate understanding of the role played by the protein aggregates in the disease and their diagnostic value, as well as perform quantitative studies from a single sample without any manual filtration and additional steps.

The AI-aided ImmunoSEIRA assures accurate prediction of the quantitative presence of oligomers 49 and fibrils 51 distinctively from mixed samples in human CSF. FIG. 6F (top) illustrates an exemplary surface functionalization scheme used to carry out measurements. FIG. 6F (top-right) shows a bar plot of two new titrated concentration mixtures of aSyn oligomers and fibrils spiked in human CSF, corresponding to (90% oligomers & 10% fibrils and 10% oligomers & 90% fibrils) at a fixed final concentration of 5 μM. FIG. 6F (middle) shows 3D spectrograms (Absorbance-Wavenumber-Timepoints) of two new mixtures that are concatenated with their corresponding labels according to the presence of fibrils (Label=10 for 90% oligomers & 10% fibrils mixture and Label=90 for 10% oligomers & 90% fibrils mixture) in each sample set. Both datasets are then split into 80-20%, respectively. After training with 80% data, the remaining 20% of testing data is fed to the newly trained network to predict the percentage presence of oligomers and fibrils in CSF. FIG. 6F (bottom) shows the results predicted by the trained DNN for these new datasets to get a comprehensive visualization of the AI-aided quantitative distinction of aSyn aggregates using ImmunoSEIRA. The comparison of the actual fibrils percentage with the predicted value by DNN from the testing data shows a good correlation with an accuracy of 92.31%. The AI-aided analysis successfully fared well in decoupling even the close-by percentages: 0-100, 10-90, 25-75% (also 100-0, 90-10, 75-25%).

The system 1 also permits multiplexed ImmunoSEIRA for simultaneous detection of aSyn and tau proteins. Increasing evidence points to the need for incorporating multiple disease-relevant biomarkers to accurately diagnose and differentiate between different NDDs. One such additional biomarker of interest in aSyn and PD studies is the tau protein because phosphorylated and aggregated forms of tau often co-occur with aSyn pathology in PD and other NDDs. Several lines of evidence point to complex interactions between aSyn and tau in PD and suggest that they influence each other's function and pathology formation. Furthermore, analysis of PD brain pathology also displayed the colocalization of tau and aSyn in LBs, which confirms tau as a complementary biomarker. Some biomarker studies have assessed the potential of simultaneously monitoring the levels of aSyn and tau in different forms and showed that it could improve the diagnosis of PD and synucleinopathies. These studies are usually based on measuring the protein levels rather than the direct assessment of their conformations and require the use of multiple assays, which increases demands on the very precious human samples. This underscores the need for a detection method capable of screening several biomarkers in a multiplexed manner.

Multiplexing was carried out and demonstrated using the ImmunoSEIRA microarray sensor 1 of the present disclosure by simultaneously detecting two different structural biomarker proteins that are for example aSyn and tau from a single sample. As previously described, the optofluidic device 17 may include a plurality of independent microfluidics channels 19, and, in each channel 19, there are provided a linearly arrayed sensing element 10 (see FIGS. 3A and 3B).

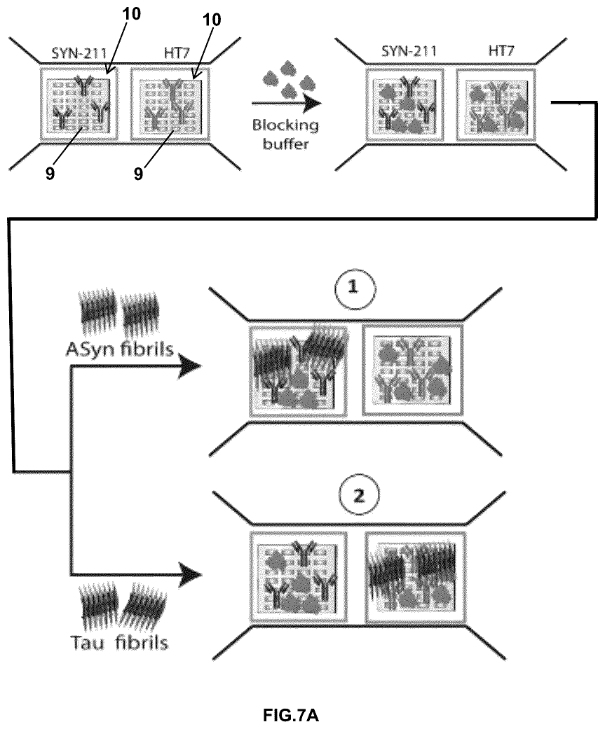

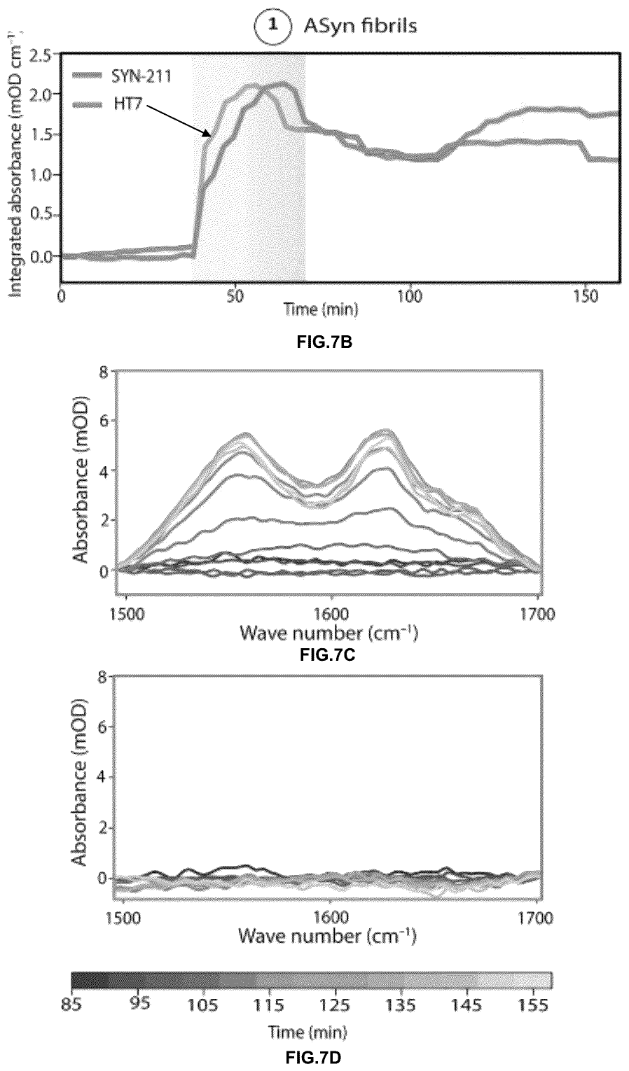

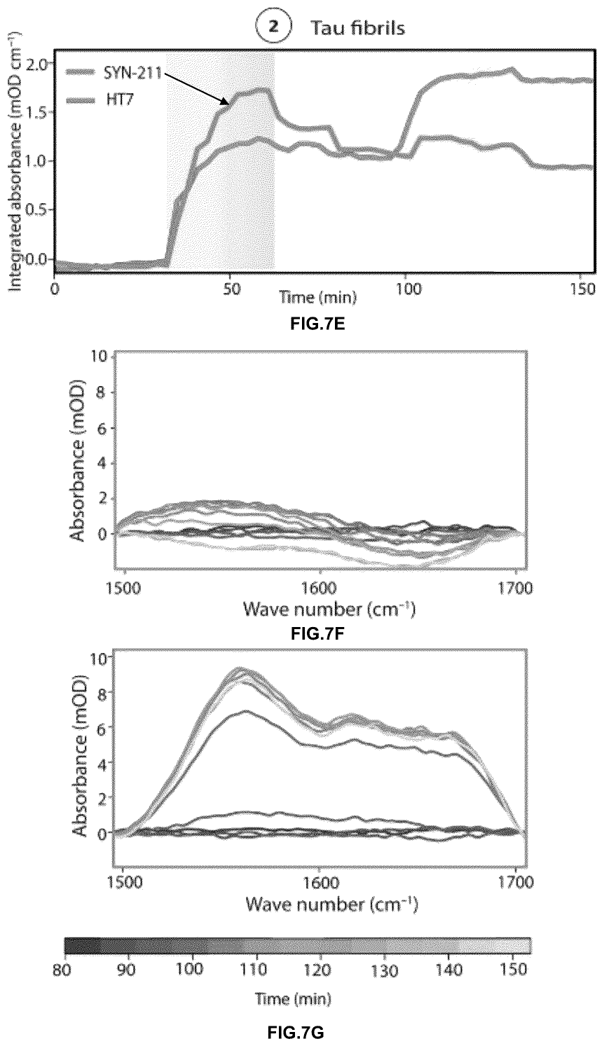

Capturing these two different proteins simultaneously requires their respective sequence-specific antibodies 15 to be selectively immobilized on the surface of the corresponding sensing regions. Spatially resolved bioprinting was performed of the capture antibodies 15 by using a noncontact and low-volume piezoelectric liquid micro-dispenser. Before antibody 15 printing, the entire surface of the plasmonic chip 3 is functionalized with activated NHS ester thiol and OH spacer thiol. The central sensing area 31 (FIG. 3A) associated the middle fluidic channel 19 is used, and a first portion thereof (for example, a first half comprising four sensing elements 10 and two mirrors 12) is functionalized with SYN-211 antibody 15 targeting aSyn and a second portion thereof (for example the second half) with HT7 antibody 15 targeting tau protein (FIG. 7A). Antibody droplets of 450-pl volume were spotted on the respective regions uniformly within a matter of minutes with high spatial precision and at low antibody consumption down to few nanoliters per sensing element.

After incubating the bioprinted chip 3 at room temperature for approximately 2 hours and washing, it is mounted to the microfluidic parts for in situ measurements. From the absorbance signals, it is confirmed that the bioprinting of both SYN-211 and HT7 antibodies 15 is highly uniform. Consequently, the light is focused on one of the bioprinted functionalized sensing elements 10 per antibody 15 and time-resolved spectral measurements are performed to establish the baseline signal. Next, the sensor surface was blocked with milk buffer to eliminate nonspecific binding sites. Afterward, the cross-reactivity of the platform is verified by injecting the proteins, aSyn fibrils and tau fibrils, each in a different experiment, and the signal change evaluated. The schematics of both experiments are shown in FIG. 7A. In the first experiment with aSyn fibrils injection (after ˜110 min), one observes (FIG. 7B) that the binding response and, thereby, the absorbance are increased in the SYN-211 spotted sensing element 10 and the sensorgram signal stabilized even after washing. On the other hand, the sensorgram signal for the HT7 spotted sensing element increased slightly with injection but, upon washing, reduced back to the previous baseline (FIG. 7B). The binding of aSyn fibrils on the SYN-211-spotted element 10 can also be observed with the retrieval of the distinct absorption signal during this injection step (FIG. 7C), and the absence of aSyn fibrils binding on the HT7 spotted element 10 is confirmed with a lack of absorption signal increase in the same duration (FIG. 6D).

In the second experiment with tau fibril injection (after ˜100 min), one observes a slight increase in the sensorgram signal on the SYN-211 spotted sensing element (FIG. 6E). But after washing (˜130 min), the signal decreased back to the previous baseline, indicating that the protein passed over the sensor surface without binding. On the other hand, in the HT7-printed sensing element, a stable binding response was observed even after washing (FIG. 7E), indicating the successful capture of tau protein by HT7 antibodies 15. This is also consistent with the lack of increase in the time-resolved absorbance spectra retrieved from the tau fibril injection step on the SYN-211 sensing element (FIG. 7F), whereas the binding of tau fibrils on the HT7 spotted array can be observed with the retrieval of the distinct absorption signal during this injection step (FIG. 7G). These results indicate that the surface is optimally blocked, and the chosen antibodies 15 are specific with no notable affinity to the other biomarker.

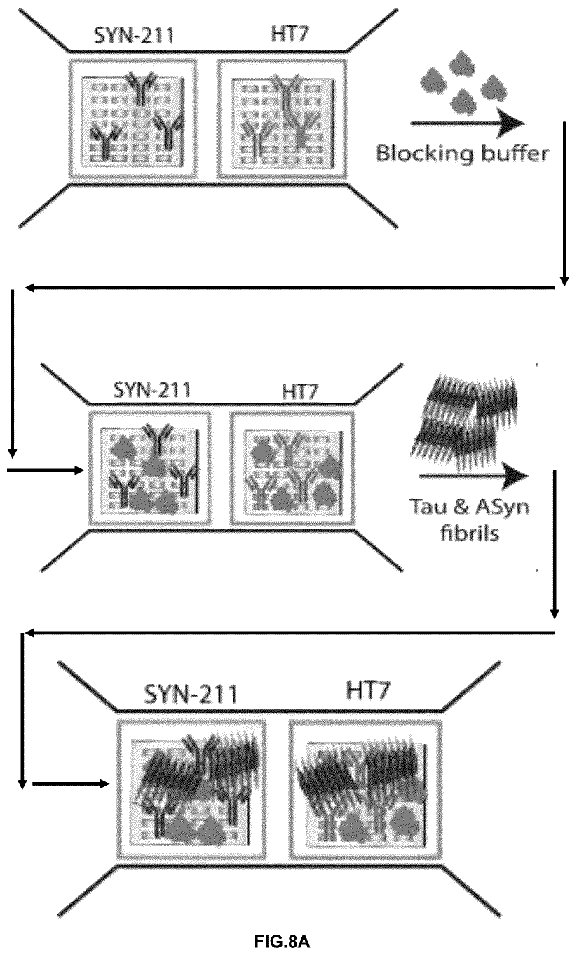

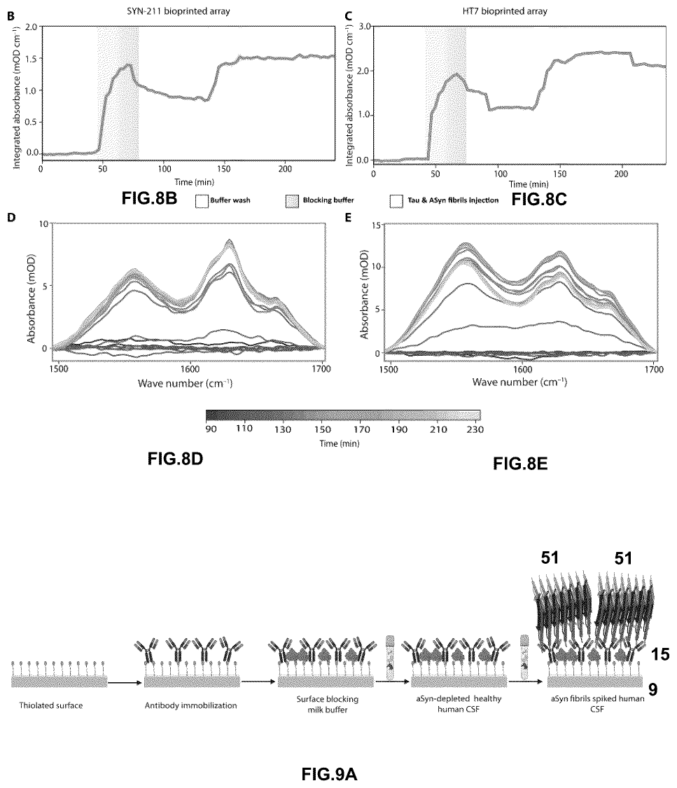

Last, for the validation of multiplexed detection of pathological tau and aSyn fibrils, a sample containing the mixture of both biomarkers was injected (FIG. 8A). Following the blocking and washing steps, a stable binding response is observed for both SYN-211- (FIG. 8B) and HT7-printed sensing elements (FIG. 8C) upon the mixture injection (˜140 min) even after the final washing step. The retrieved time-resolved absorption spectra also show a stable signal increase and capture distinct signatures of aSyn and tau fibrils on the SYN-211- (FIG. 8D) and HT7-printed sensing elements (FIG. 8E), respectively.

To take the developed sensor one step closer to clinical application, it is important to assess its detection abilities in a complex biomatrix, and it the retrieval of the absorption signature of aSyn fibrils in a complex biomatrix is now demonstrated.

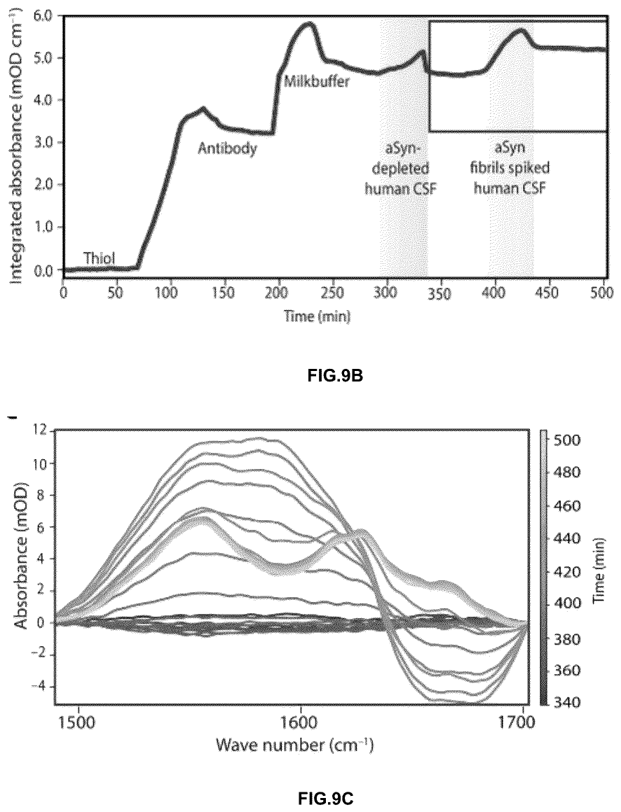

The transitioning of the operation from buffer to real and complex human body fluids is not straightforward. There are several differences between analyte studies in a buffer system to that in its native body fluid environment. For example, the physiological response of the analytes, such as their binding affinity and structural integrity, can be different. In addition, the presence of other biomolecular components in the body fluids can lead to biofouling, thus having an interfering effect with the target capture and detection. These factors need to be carefully assessed and dealt with for the successful application of clinical samples. To illustrate the feasibility of the sensor 1, healthy human CSF samples depleted of aSyn by an in-house optimized protocol were used as the biomatrix. It was observed that milk buffer is a suitable blocking agent to minimize nonspecific binding from the molecules/proteins present in the biomatrix. The schematic of the experimental procedure is shown in FIG. 9A. After the antibody and blocking steps, a negative control is performed by injecting aSyn-depleted human CSF (˜290 min). Initially, the sensorgram signal is increased, indicating the presence of multiple nonspecific molecules present in it. But after the washing buffer was introduced (˜340 min), the signal returned to the previous baseline, confirming that the surface is immune to biofouling and to the binding of other molecules in depleted CSF (FIG. 9B). Last, when it was injected a 200-μl sample of aSyn fibrils (5 μM) spiked in depleted CSF matrix (˜390 min) for enriching the protein capture on the sensor surface, a stable sensorgram signal increase is observed even after a washing step (˜440 min), indicating specific binding of aSyn fibrils on the functionalized surface. Also shown is the absorbance spectra during the injection step in FIG. 9C, which demonstrates successful retrieval of the aSyn fibrils absorption signature in the presence of the complex biomatrix.

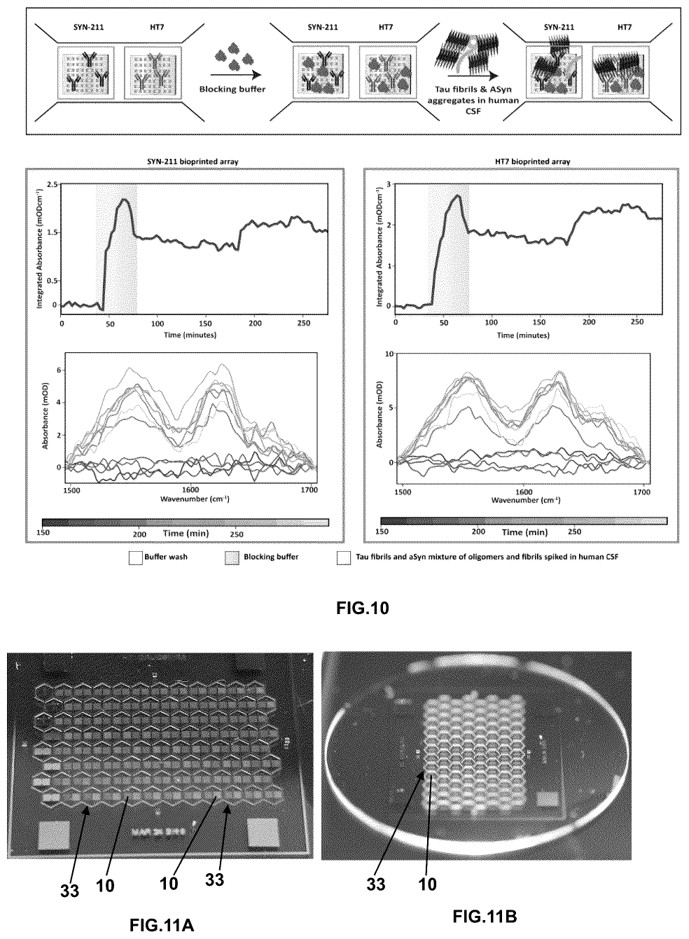

Furthermore, proof-of-concept experiments combining different strengths of ImmunoSEIRA were performed, including multiplexing, AI-aided analysis, measuring aSyn aggregate mixture in different ratios and operation in complex biomatrix (see FIGS. 10 and 6F), which paves the way to use the method and system 1 of the present disclosure in scientific research on understanding NDDs and toward clinical studies.

As set out above, the unique capabilities of SEIRA have been assessed for its potential as a diagnostic tool by structural analysis of protein biomarkers. The detection principle of SEIRA in PIR configuration provides extreme field confinement close to the nanoantennas 9, thereby overcoming the problem of water absorption obscuring the protein signal. This subsequently enabled straightforward and easy integration of SEIRA substrate 3 with microfluidics 17 to perform in situ detection from a small sample volume while maintaining the conformational integrity of the proteins, in contrast to other IR methods like grazing incidence reflection.

An incorporated immunoassay with SEIRA (ImmunoSEIRA) is provided for conformationally sensitive and label-free analysis of structural biomarkers of the NDD-relevant proteins aSyn and tau, by directly accessing their distinct chemical vibrational fingerprints. With this immunoSEIRA sensor 1, comprehensive structural profiling is performed of all three conformational species of aSyn associated with pathology formation in PD and other NDDs, including monomers, oligomers, and fibrils. Different structural motifs present in each of the conformational aSyn species successfully quantified and the differences and similarities between them were identified.

In an unprecedented manner, by combining ImmunoSEIRA with AI, it was showed that one could not only differentiate between different aggregations states of aSyn with distinct conformational properties but also quantitatively predict the individual presence of oligomers and fibrils from mixed aSyn aggregate samples. This outcome is a major advancement from current assays, which depend on antibodies that cannot reliably distinguish between oligomers and fibrils. The AI-aided ImmunoSEIRA analysis is crucial for extensively profiling the quantitative presence of different conformational species of the same protein biomarker in patient body fluids. This can lead to developing a structural fingerprint map of protein variations occurring from the early prodromal stage until the late clinical stage to understand in-depth the potential role of aSyn in disease pathology and as a clinical biomarker.

Also demonstrated is the sensor 1 forming a multiplexed SEIRA microarray that can incorporate and detect multiple pathological markers of NDDs, e.g., tau and aSyn fibrils, simultaneously. Existing efforts for multiple biomarker detection using IR methods either use different sensors for each biomarker or regenerate the same sensor by repetitive and laborious functionalization steps. Crucially, these approaches require the repetitive use of higher amounts of precious biofluids, which is not clinically feasible considering the total volume of extracted sample (e.g., a few ml of CSF) and the different types of tests and repetitions of measurements to be done. These limitations are overcome by exploiting the inherent 2D microarray design format of the sensor 1 and performing multiplexed detection of two protein biomarkers from a single low-volume sample injection of 200 μl, which could be brought down further. These multiplexing capabilities provide unique opportunities for the assessment of multiple disease-relevant biomarkers, including multiple proteins linked to NDDs. Recent studies have shown that the presence of pathological aggregates of multiple proteins, including amyloid-beta, aSyn, tau, and TDP-43, is the norm rather than the exception in NDDs and that the relative distribution of aggregates of these proteins in the brain influences disease symptomology and rate of progression.

One alternative use of the multiplexed SEIRA microarray is to use multiple antibodies of the same protein to capture the diversity of aggregates in biological samples. The availability of species-specific antibodies would substantially increase the diagnostic power of our AI-aided ImmunoSEIRA as it will allow more precise determination of the ratios of different species (monomers, oligomers, fibrils, or post-translationally modified forms of these proteins), which could be combined with other biomarkers to enable more accurate assays for early detection and monitoring disease progression.

The performance of the sensor 1 was evaluated for conformational sensing of aSyn fibrils when spiked in healthy human CSF sample and demonstrated its structural fingerprint retrieval even in the presence of compounded signals from the complex biomatrix. These observations represent proof-of-concept results paving the way to test and validate the ImmunoSEIRA assay in body fluids from cohorts of patients at different stages of PD and patients with different synucleinopathies.

Further details of the measurements performed and exemplary embodiments of the elements of the system 1 from which the measured results are obtained are now presented.

For the numerical simulation of the plasmonic metasurfaces of the nanostructures 9, a finite integration Maxwell solver CST Studio 2018 is used for the numerical design and characterization of the far-field and near-field properties of the nanostructures. A unit cell consisting of a nanorod 9 with a height of 100 nm of Au (gold) over 5 nm of Cr (chromium) is created on calcium difluoride (CaF₂) substrate with a water layer on top of the antennas with optical constants retrieved from Olmon et al. (reference 81), Rakić et al. (reference 82), Li (reference 83), and Hale et al. (reference 84) for Au, Cr, CaF₂, and water respectively. The structures are modeled with a tetrahedral mesh of 40-nm size and simulated with periodic boundary conditions. The incident plane wave is set to have an inclination angle of 16.7° as an average of the incident angle spread (9.8° to) 23.6° with the Cassegrain objective used in the FTIR measurements. The far-field spectrum is calculated as the average of the transverse magnetic (TM) excitation mode perpendicular to the long edges of the nanorod and the transverse electric (TE) excitation mode perpendicular to the short edges.

For the fabrication of the plasmonic metasurfaces of the nanostructures 9, CaF₂ chips of 13-mm diameter and 0.5-mm thickness are used as the substrates (Crystran, UK). After RCA1 (NH₄OH:H₂O₂:H₂O=1:1:5) cleaning protocol and subsequent washing with acetone and isopropyl alcohol (IPA), chips are spin-coated with low- and high-molecular weight PMMA [poly(methyl methacrylate)]. This is sputtered with 10 nm of Au as a conductive layer for electronic beam lithography. The nanorod arrays of 250×250 μm² are patterned with 5-nm resolution and Au mirrors of the same size with 100-nm resolution using a 100-keV beam. Afterward, the Au layer is removed by wet etching with KI+I2 solution and subsequently developed with a PMMA developer MiBK:IPA=1:3. Then, electron beam evaporation is done to deposit 5 nm of Cr with 100 nm Au. Liftoff is done with acetone and followed by heated sonication in MICROPOSIT REMOVER 1165 and water to remove the resist residues completely. Last, structures are analyzed using scanning electron microscopy to verify the successful fabrication. The nanorods 9 used in the experiments are 1500 nm in length, 100 nm in width and height in an array with a y-axis periodicity of 3200 nm and a gap of 80 nm between the nanorods 9 in the x direction.

For the fabrication of microfluidic micro-flowcell 18 and chipcell 27, the upper layer of the micro-flowcell 18 with small channels of width (300 μm) and depth (30 μm) is fabricated using standard soft lithography. The Si wafer is coated with a positive resist (AZ1512) and exposed via direct laser writing. After development, the wafer is dry-etched (Bosch process), followed by resist stripping to form the mold. The mold for the chipcell 27 and the big channel layer of the micro-flowcell 18 are created using a vinyl-based cutting plotter (GRAPHTEC). All the molds are silanized for PDMS fabrication by incubating them with TMCS (trimethylchlorosilane). Then, the mixture of PDMS and curing agent in the ratio of 10:1 is poured over the mold surfaces and baked at 80° C. for over 2 hours. The upper and big channels for the micro-flowcell 18 are then oxygen plasma-bonded with alignment to form the complete micro-flowcell part.

For the immunoassay protocol, the sensor chip 3 is washed with acetone, ethanol, and water, followed by oxygen plasma cleaning. Afterward, within 30 min, it is incubated with a 2 mM thiol mixture of activated ester (HSC11EG4OCH2COONHS, ProChimia Surfaces) and spacer thiols (HS-C6-EG3OH, ProChimia Surfaces) in the ratio of 1:9 overnight. After washing with ethanol, the chip 3 is mounted with the microfluidic parts 27, 18 to start the in-situ immunoassay steps. With the continuous 1×PBS [10 mM phosphate buffer, 2.7 mM potassium chloride, and 137 mM sodium chloride (pH 7.4)] buffer flow started, 200-μl SYN-211 antibody (40 μg/ml) (#ab206675, Abcam; sc-12767, Santa Cruz Biotechnology) in sodium acetate buffer is injected. Once the antibody signal is stabilized after washing, 10 μM of 200-μl aSyn fibrils (in-house prepared) is injected. In the aSyn oligomer capture (FIG. 4D) and the monomer capture (FIG. 4E) experiments, 200 μl of protein solutions of 5 μM oligomers and 10 μM monomers are used, respectively.

For the Infrared in situ measurements, the in-situ measurements are carried out using an FTIR spectrometer (Bruker Vertex 80v) coupled to a microscope (HYPERION 3000 IR microscope) with a reflective Cassegrain objective (15×, numerical aperture=0.4), with high-power globar as the light source and liquid nitrogen-cooled mercury cadmium telluride as the detector. An external polarizer is used to apply incident light polarization parallel to the long axis of the nanorods. A knife-edge aperture limits the light collection to slightly less than 250×250 μm², which is the size of each sensing element and mirror. The measurements are done in reflection mode, illuminating the sensor chip from the backside of the CaF₂ substrate to avoid light propagation through buffer/water and in a purged dry air environment. The inlet of the microfluidic parts is connected to an external pump coupled with a flow rate sensor to precisely control the flow rate of buffer and analyte injection. The buffer is run continuously at a flow rate of 50 μl/min, and analytes are injected onto the sensor surface at 10 μl/min. For collecting data, one of the sensing elements in the middle fluidic channel and its neighboring mirror is chosen for sensing and referencing, respectively. The in-flow experiments are done by measuring the mirror once, followed by 20 times the sensing element in a continuous loop, with 32 scans per measurement. For the experiments described in FIG. 4 for the secondary structure analysis, measurements are done with 512 scans with repetitive loops of one mirror followed by four times the sensing element measurements.

For the FTIR data analysis and absorbance calculation, the continuous extraction of the protein absorbance fingerprints in the amide range (1500 to 1700 cm⁻¹) is carried out by normalizing the spectrally averaged reflectance spectra obtained following the protein injection (R_{NDD}) to that of the spectra of the reference sensor in the buffer in the previous functionalization state (R₀). Then, the differential absorbance spectrum in mOD is retrieved using the formula—1000*log 10 (R_{NDD}/R₀). A baseline correction procedure is performed on the obtained data before displaying the final absorbance spectra curves. This is done by subtracting a second-order polynomial fitted to the absorption spectra in the range of 1450 to 1700 cm⁻¹. This extracted amide band absorbance is then integrated over the range 1525 to 1650 cm⁻¹ to output the integrated time absorbance plots used to monitor the binding kinetics.

For secondary structure analysis, the secondary structure analysis of the aSyn species shown in FIG. 5B follows the protocol from Yang et al. (reference 56). The reflectance spectra are collected with 512 scans per measurement and 4-cm⁻¹ spectral resolution. Two hundred microliters of 5 μM aSyn fibrils, 5 M aSyn oligomers, and 10 μM aSyn monomers are used in separate experiments for this analysis. The retrieved absorbance spectra after the stabilized protein binding are used to execute the secondary structure analysis. First, a second-derivative analysis (a rate of change of [a rate of change of an absorption value over a spectral range of the absorption spectra]) is performed on the absorbance spectra to separate the overlapping bands without any execution bias. This is done by applying a second-order Savitzy-Golay filter with a seven-point calculation window. The number of subbands and their peak position data obtained from this analysis are then used to resolve the secondary structure information using FSD. The absorbance spectra is fitted as a linear combination of Lorentzian/Gaussian curves with the peak positions the same as the frequencies obtained from the second-derivative analysis through multiple iterations until convergence is reached. Last, the area under each curve is integrated to calculate each subband's relative contribution, corresponding to the percentage presence of a particular secondary structure motif.

For the AI-DNN analysis, the spectra for the entire dataset used in AI-DNN analysis are obtained as mentioned above. The stock concentration of the in-house prepared oligomers and fibrils is measured using bicinchoninic acid (BCA) assay and NanoDrop measurements. The mixtures of the aggregates of different percentage combinations used in the analysis are carefully titrated from these stock protein solutions with a fixed final mixture concentration of 5 μM. In total, seven mixtures with oligomers and fibrils were prepared in the following relative percentages of each (oligomers-fibrils): 100-0, 75-25, 60-40, 50-50, 40-60, 25-75, and 0-100%. The same protocol is followed as for the Immunoassay protocol. After stabilizing the integrated sensorgram signal from antibody binding [200 μl, SYN-211 (40 μg/ml)] followed by washing, 200 μl of the aggregate mixture is injected. The stable absorbance of the antibody is taken as a baseline and measuring the spectra of the injected mixture and the corresponding binding on the surface is started. The absorbance spectra in the wave number range of 1450 to 1700 cm⁻¹ are continuously monitored until the mixture flows over the surface and is washed. The collected raw absorbance spectra are used without any spectral averaging (i.e., one spectral line shown in FIG. 6 for each mixture corresponds to one 32-scan measurement). For uniformity, 350 such spectra were selected for each mixture that starts from the time of injection and up to the point of stabilization. This gives a dataset of each mixture with 350 spectra of different time points with 133 data points of absorbance over the wave number range of 1450 to 1700 cm⁻¹. For each mixture, every spectrum involved is labeled with the value that corresponds to the percentage presence of fibrils (FIG. 6E). The absorbance plots retrieved and their labels (shown in FIG. 6) for each mixture are used as the entire training/testing dataset for the AI model. The AI-DNN model is created in Python language using the scikit-learn library. A DNN is created based on the multi-layer perceptron MLP model as this was treated as a regression problem.

To match with the input data points, the DNN model has 133 input nodes corresponding to each wave number point that will take in/fed by the absorbance value for that particular wave number at a given time point and 1 output node to feed/output the corresponding label of the spectra. To optimize and tune the parameters of the model, like the number of hidden layers and the total number of nodes per hidden layer, the K-fold CV method was used. For this purpose, the entire dataset was split randomly into 80 and 20% (i.e., the total spectra of each mixture). The 80% of the thus collected dataset is then used for the training and K-fold CV. A fivefold CV was executed on the training dataset using the solver Adam and the activation function as logistic. The loss function was selected to be the mean squared error for the optimization step to yield the appropriate parameters leading to the least mean squared error possible. The parameters of 4 hidden layers with 26 nodes each are obtained from this training step. The rest of the 20% of the dataset is then fed to this optimized MLP-DNN model [depth (h)=5, width (dₘ)=26, and input dimension (n)=133], and the predicted output labels from the network for each spectrum of every mixture ratios are plotted against the actual label (percentage concentration of fibrils) in the plot shown in FIG. 6D.

For the multiplexing experiment, the sensor chip 1 is incubated with a 2 mM thiol mixture of activated ester (HSC11EG4OCH2COONHS, ProChimia Surfaces) and spacer thiols (HS-C6-EG3OH, ProChimia Surfaces) in the ratio of 1:9 overnight. Afterward, the chip 3 is washed with ethanol to wash away unbound thiols and dried. Without any delay, the chip 3 is put in a petri dish and placed inside the tool sciFLEXARRAYER S3 from Scienion (piezoelectric noncontact ultralow-volume dispensing system). The dispensing system is set to be on humidity control at dew point (66% relative humidity) and internal temperature of 16° C. to prevent evaporation of the spotted solution. The bioprinting solutions consist of 50 μg/ml each of SYN-211 and HT7 antibodies separately in 10 mM sodium acetate buffer. The middle sensor linear array 31 is focused using the camera, and then one-half of the array containing four sensing elements 10 and two mirrors 12 are dispensed with multiple microarray spots of 450 μl of SYN-211 solution and the other half with HT7 solution, respectively. Water droplets are pipetted inside the petri dish surrounding the chip, and the lid is closed and then covered with paraffin film to prevent evaporation. Then, the chip 3 is incubated at room temperature for approximately 2 hours. Afterward, the chip 3 is washed with 1×PBST (PBS containing 0.1% Tween 20) solution, followed by water, and dried. Afterward, the chip 3 is placed on the microfluidic chipcell 27 and incorporated with the micro-flowcell 18 to start the in-flow SEIRA measurements. To eliminate nonspecific binding, the sensor 1 is blocked using 300 μl of 2× milk buffer [Pierce Clear Milk Blocking Buffer (10×), Thermo Fisher Scientific]. For the cross-reactivity experiments shown in FIG. 7, 5 μM of 200-μl aSyn fibrils or 5 μM of 200-μl tau fibrils were used. For the combination injection step in the experiment shown in FIG. 8, the 200-μl sample contained an equal amount of aSyn fibrils and taufibrils, each at a total concentration of 5 μM.

(000174) In relation to the in-vitro preparation of aSyn structural species, for monomers, recombinant overexpression and purification of human wild-type (WT) aSyn monomers were purified as previously reported (see reference 53). pT7-7 plasmids encoding human WT aSyn were used for transformation in BL21 (DE3) Escherichia coli cells on an ampicillin agar plate. A single colony was transferred to 400 ml of LB medium comprising ampicillin (100 μg/ml; AppliChem, A0839), followed by overnight incubation at 37° C. and 180 rpm. As a next step, the preculture was used to inoculate 3 to 6 liters of LB medium, including ampicillin (100 μg/ml). The induction of aSyn protein expression was further performed upon A600 forthcoming 0.4 to 0.6 via adding 1 mM 1-thio-β-d-galactopyranoside (AppliChem, A1008). Next, the cells were incubated at 37° C. and 180 rpm for 4 hours, followed by centrifugation at 4000 rpm at 5° C. for 30 min, using JLA 8.1000 rotor (Beckman Coulter, Bear, CA, USA). The resulting pellets were stored at −20° C. until further steps. The cell lysis was conducted by dissolving the resulting pellet in 20 mM Tris-HCl (pH 7.5) containing protease inhibitors [1 mM EDTA (Sigma-Aldrich, 11,873,580,001] and 1 mM phenylmethylsulfonyl fluoride (Applichem, A0999) (buffer A), which was ultrasonicated (VibraCell VCX130, Sonics, Newtown, CT, USA) using the following conditions: time: 5 min; cycle: 30 s ON, 30 s OFF; amplitude: 70%. As a next step, the samples were centrifugated for 30 min at 12,000 rpm and 4° C. for 30 min, and the supernatant was collected in a 50-ml Falcon tube and further located in boiling water (100° C.) for approximately 15 min. The protein sample was subsequently centrifuged similarly to the abovementioned conditions, and the resultant supernatant was filtered through 0.45-μm filters and injected into a sample loop connected to HiPrep Q FF 16/10 (GE Healthcare). The supernatant was injected at 2 ml/min and eluted using 20 mM tris-HCl, 1 M NaCl (pH 7.5) (buffer B) from gradient 0 to 70% at 3 ml/min. All fractions were analyzed by SDS-polyacrylamide gel electrophoresis (PAGE), and the positive pure aSyn was pooled. For the aSyn monomers prepared for using monomers as such and for the preparation of fibrils, further purification is done by reverse-phase high-performance liquid chromatography (Jupiter 300 C4, 20 mm I.D.×250 mm, 10-μm average bead diameter, Phenomenex) and lyophilized. Toward the oligomer preparation, after the HiPrep test, the aSyn-positive samples are pooled and dialyzed against deionized water at 4° C. overnight to remove salts and subsequently snap-frozen and lyophilized.

For unmodified oligomers, unmodified WT aSyn oligomers were prepared as previously described (see reference 53). Briefly, 60 mg of lyophilized protein was dissolved in 5 ml of PBS buffer (pH 7.4) and incubated at 900 rpm with constant shaking at 37° C. for 5 hours. The sample was centrifuged at 12,000 g at 4° C. for 10 min, aiming to remove any insoluble species. As a next step, the supernatant was loaded onto a HiLoad 26/600 Superdex 200 preparation grade (GE Lifesciences) column pre-equilibrated with PBS buffer (pH 7.4). Protein was eluted as 2.5-ml fractions at a flow rate of 1 ml/min. All fractions were further analyzed by SDS-PAGE, and the positive oligomer fractions (i.e., void volume peak) were snap-frozen and stored at −20° C. for further analyses.

For fibrils, WT aSyn fibrils were similarly prepared as previously stated (see references 8, 53). Briefly, 4 mg of aSyn monomers was diluted in 600 μl of PBS (pH 7.4), and the pH was adjusted to ˜7.2 to 7.4. After, the monomeric aSyn solution was filtered through 0.2-μm filters (Merck, SLGP033RS) before being incubated under constant orbital agitation (1000 rpm) at 37° C. for 5 days. The extent of the formation of fibrils was assessed by SDS-PAGE and negative-staining TEM. aSyn WT fibrils were further sonicated on ice with a fine tip (Sonics Vibra cell) for 20 s, at 20% amplitude, a pulse of 1 s ON/1 s OFF (Sonic Vibra-Cell, Blanc-Labo, Switzerland). The number of monomers and oligomers released from sonicated fibrils was quantified using a filtration protocol. Sonicated aSyn fibrils were characterized by TEM and further aliquoted, snap-frozen, and stored at −80° C. until the subsequent analyses.

Concerning SDS-PAGE and Western blot analysis, for SDS-PAGE analysis, Human WT aSyn monomers, oligomers, and fibrils were mixed with 5× Laemmli buffer and subsequently loaded onto 15% polyacrylamide gels. The gel was run at 180 V for 1 hour, and the protein bands were visualized by Coomassie's brilliant blue staining. Human WT aSyn monomers, oligomers, and fibril samples were also analyzed and characterized through Western blot. The aSyn species samples were loaded and separated on 15% polyacrylamide gels and run under similar conditions as the abovementioned. Subsequently, the proteins were transferred onto a nitrocellulose membrane (Thermo Fisher Scientific, Switzerland) using a semi-dry system (Bio-Rad, Switzerland) at 25 V, 0.5 A, and 45 min. The transferred proteins were further blocked for 1 hour using Odyssey blocking buffer [Li-Cor, Lincoln, NE, USA, (P/N: 927-40000)] and incubated overnight at 4° C. with the primary antibody SYN-1 (#610787, BD Biosciences). On the next day, the membranes were washed three times in 1×PBST for 10 min at room temperature and incubated with IR dye-conjugated secondary antibodies for 1 hour protected from light also at room temperature. After, the membranes were washed three times in a similar way as aforesaid. Last, protein bands were visualized by fluorescence imaging using the Odyssey CLx System (Li-Cor, NE, USA).

For aSyn depletion protocol from healthy human CSF, the immunoprecipitation assay was performed using a Dynabeads Antibody coupling kit (Invitrogen, USA) with superparamagnetic Dynabeads M-270 Epoxy beads following the manufacturer's instructions. Briefly, 2 to 5 μg of mouse antibody (#848302, BioLegend, USA) was mixed with 1 mg of magnetic beads and incubated overnight at 37° C. On the next day, 1 ml of crude Human CSF samples was thawed on ice, followed by the addition of protease and phosphatase inhibitors. As a first step, a preclearing of the CSF sample was conducted to decrease/deplete the levels of endogenous immunoglobulin Gs (IgGs) by mixing the CSF with Pierce Protein G Magnetic Beads (Thermo Fisher Scientific, USA) for 2 hours at 4° C. The resulting antibody-conjugated epoxy beads were mixed with the IgG depleted CSF sample and incubated overnight on a rocking platform at 4° C. As a next step, the magnetic beads/sample solution was transferred to a magnetic particle processor, and the supernatant was collected (unbound fraction), which is the aSyn-depleted CSF used in the experiment shown in FIG. 9 as the complex biomatrix. The antibody-conjugated epoxy beads were washed twice with 1×PBST and once with PBS (pH 7.4). aSyn was eluted by adding a solution based on 50% acetonitrile/50% water/0.1% TFA (IP sample). The IP'ed CSF sample was dried in a SpeedVac and resuspended in PBS (pH 7.4).

While the invention has been disclosed with reference to certain preferred embodiments, numerous modifications, alterations, and changes to the described embodiments, and equivalents thereof, are possible without departing from the sphere and scope of the invention. Accordingly, it is intended that the invention not be limited to the described embodiments and be given the broadest reasonable interpretation in accordance with the language of the appended claims. The features of any one of the above described embodiments may be included in any other embodiment described herein.

### REFERENCES

1. C. Soto, Unfolding the role of protein misfolding in neurodegenerative diseases. Nat. Rev. Neurosci. 4, 49-60 (2003).
2. C. A. Ross, M. A. Poirier, Protein aggregation and neurodegenerative disease. Nat. Med. 10, S10-S17 (2004).
3. C. Foguem, P. Manckoundia, Lewy body disease: Clinical and pathological “Overlap Syn-drome” between synucleinopathies (Parkinson disease) and tauopathies (Alzheimer disease). Curr. Neurol. Neurosci. Rep. 18, 24 (2018).
4. M. G. Spillantini, M. L. Schmidt, V. M. Y. Lee, J. Q. Trojanowski, R. Jakes, M. Goedert, α-Synuclein in Lewy bodies. Nature 388, 839-840 (1997).
5. H. A. Lashuel, Do Lewy bodies contain alpha-synuclein fibrils? And does it matter? A brief history and critical analysis of recent reports. Neurobiol. Dis. 141, 104876 (2020).
6. H. Braak, K. D. Tredici, U. Rub, R. A. I. deVos, E. N. H. Jansen Steur, E. Braak, Staging of brain pathology related to sporadic Parkinson's disease. Neurobiol. Aging 24, 197-211 (2003).
7. D. J. Moore, A. B. West, V. L. Dawson, T. M. Dawson, Molecular pathophysiology of Parkinson's disease. Annu. Rev. Neurosci. 28, 57-87 (2005).
8. A. L. Mahul-Mellier, J. Burtscher, N. Maharjan, L. Weerens, M. Croisier, F. Kuttler, M. Leleu, G. W. Knott, H. A. Lashuel, The process of Lewy body formation, rather than simply α-synuclein fibrillization, is one of the major drivers of neurodegeneration. Proc. Natl. Acad. Sci. U.S.A. 117, 4971-4982 (2020).
9. H. A. Lashuel, C. R. Overk, A. Oueslati, E. Masliah, The many faces of α-synuclein: From structure and toxicity to therapeutic target. Nat. Rev. Neurosci. 14, 38-48 (2013).
10. K. A. Conway, S. J. Lee, J. C. Rochet, T. T. Ding, R. E. Williamson, P. T. Lansbury Jr., Acceleration of oligomerization, not fibrillization, is a shared property of both α-synuclein mutations linked to early-onset Parkinson's disease: Implications for pathogenesis and therapy. Proc. Natl. Acad. Sci. U.S.A. 97, 571-576 (2000).
11. J. Hardy, G. Higgins, Alzheimer's disease: The amyloid cascade hypothesis. Science 256, 184-185 (1992).
12. C. Ballatore, V. M. Y. Lee, J. Q. Trojanowski, Tau-mediated neurodegeneration in Alzheimer's disease and related disorders. Nat. Rev. Neurosci. 8, 663-672 (2007).
13. L. Söderberg, M. Johannesson, P. Nygren, H. Laudon, F. Eriksson, G. Osswald, C. Möller, L. Lannfelt, Lecanemab, aducanumab, and gantenerumab-Binding profiles to different forms of amyloid-beta might explain efficacy and side effects in clinical trials for Alzheimer's disease. Neurotherapeutics 20, 195-206 (2023).
14. B. Mollenhauer, J. J. Locascio, W. Schulz-Schaeffer, F. Sixel-Döring, C. Trenkwalder, M. G. Schlossmacher, α-Synuclein and tau concentrations in cerebrospinal fluid of patients presenting with parkinsonism: A cohort study. Lancet Neurolo. 10, 230-240 (2011).
15. L. M. Shaw, H. Vanderstichele, M. Knapik-Czajka, C. M. Clark, P. S. Aisen, R. C. Petersen, K. Blennow, H. Soares, A. Simon, P. Lewczuk, R. Dean, E. Siemers, W. Potter, V. M. Y. Lee, J. Q. Trojanowski; Alzheimer's Disease Neuroimaging Initiative, Cerebrospinal fluid bio-marker signature in Alzheimer's disease neuroimaging initiative subjects. Ann. Neurol. 65, 403-413 (2009).
16. A. Nakamura, N. Kaneko, V. L. Villemagne, T. Kato, J. Doecke, V. Doré, C. Fowler, Q. X. Li, R. Martins, C. Rowe, T. Tomita, K. Matsuzaki, K. Ishii, K. Ishii, Y. Arahata, S. Iwamoto, K. Ito, K. Tanaka, C. L. Masters, K. Yanagisawa, High performance plasma amyloid-β biomarkers for Alzheimer's disease. Nature 554, 249-254 (2018).
17. P. Magalhães, H. A. Lashuel, Opportunities and challenges of alpha-synuclein as a potential biomarker for Parkinson's disease and other synucleinopathies. NPJ Parkinsons Dis. 8, 93 (2022).
18. G. Vivacqua, A. Latorre, A. Suppa, M. Nardi, S. Pietracupa, R. Mancinelli, G. Fabbrini, C. Colosimo, E. Gaudio, A. Berardelli, Abnormal salivary total and oligomeric alpha-synu-clein in Parkinson's disease. PLOS ONE 11, e0151156 (2016).
19. P. L. McGeer, M. Lee, K. Kennedy, E. G. McGeer, Saliva diagnosis as a disease predictor. J. Clin. Med. 9, 377 (2020).
20. P. Bhattacharjee, A. Öhrfelt, T. Lashley, K. Blennow, A. Brinkmalm, H. Zetterberg, Mass spectrometric analysis of Lewy body-enriched α-synuclein in Parkinson's disease. J. Proteome Res. 18, 2109-2120 (2019).
21. A. Viodé, S. Epelbaum, I. Benyounes, M. Verny, B. Dubois, C. Junot, F. Fenaille, F. Lamari, F. Becher, Simultaneous quantification of tau and α-synuclein in cerebrospinal fluid by high-resolution mass spectrometry for differentiation of Lewy body dementia from Alzheimer's disease and controls. Analyst 144, 6342-6351 (2019).
22. L. Malinovska, V. Cappelletti, D. Kohler, I. Piazza, T. H. Tsai, M. Pepelnjak, P. Stalder, C. Dörig, F. Sesterhenn, F. Elsässer, L. Kralickova, N. Beaton, L. Reiter, N. de Souza, O. Vitek, P. Picotti, Proteome-wide structural changes measured with limited proteolysis-mass spectrometry: An advanced protocol for high-throughput applications. Nat. Protoc. 18, 659-682 (2023).
23. M. T. Mackmull, L. Nagel, F. Sesterhenn, J. Muntel, J. Grossbach, P. Stalder, R. Bruderer, L. Reiter, W. D. J. van de Berg, N. de Souza, A. Beyer, P. Picotti, Global, in situ analysis of the structural proteome in individuals with Parkinson's disease to identify a new class of bio-marker. Nat. Struct. Mol. Biol. 29, 978-989 (2022).
24. F. van Diggelen, D. Hrle, M. Apetri, G. Christiansen, G. Rammes, A. Tepper, D. E. Otzen, Two conformationally distinct α-synuclein oligomers share common epitopes and the ability to impair long-term potentiation. PLOS ONE 14, e0213663 (2019).
25. N. N. Vaikath, N. K. Majbour, K. E. Paleologou, M. T. Ardah, E. vanDam, W. D. J. vandeBerg, S. L. Forrest, L. Parkkinen, W. P. Gai, N. Hattori, M. Takanashi, S. J. Lee, D. M. A. Mann, Y. Imai, G. M. Halliday, J. Y. Li, O. M. A. El-Agnaf, Generation and characterization of novel con-formation-specific monoclonal antibodies for α-synuclein pathology. Neurobiol. Dis. 79, 81-99 (2015).
26. D. J. Covell, J. L. Robinson, R. S. Akhtar, M. Grossman, D. Weintraub, H. M. Bucklin, R. M. Pitkin, D. Riddle, A. Yousef, J. Q. Trojanowski, V. M. Y. Lee, Novel conformation-selective alpha-synuclein antibodies raised against different in vitro fibril forms show distinct patterns of Lewy pathology in Parkinson's disease. Neuropathol. Appl. Neurobiol. 43, 604-620 (2017).
27. G. G. Kovacs, U. Wagner, B. Dumont, M. Pikkarainen, A. A. Osman, N. Streichenberger, I. Leisser, J. Verchère, T. Baron, I. Alafuzoff, H. Budka, A. Perret-Liaudet, I. Lachmann, An antibody with high reactivity for disease-associated α-synuclein reveals extensive brain pathology. Acta Neuropathol. 124, 37-50 (2012).
28. S. T. Kumar, S. Jagannath, C. Francois, H. Vanderstichele, E. Stoops, H. A. Lashuel, How specific are the conformation-specific α-synuclein antibodies? Characterization and vali-dation of 16 α-synuclein conformation-specific antibodies using well-characterized preparations of α-synuclein monomers, fibrils and oligomers with distinct structures and morphology. Neurobiol. Dis. 146, 105086 (2020).
29. F. X. Theillet, A. Binolfi, B. Bekei, A. Martorana, H. M. Rose, M. Stuiver, S. Verzini, D. Lorenz, M. van Rossum, D. Goldfarb, P. Selenko, Structural disorder of monomeric α-synuclein persists in mammalian cells. Nature 530, 45-50 (2016)
30. Y. Xiao, I. Matsuda, M. Inoue, T. Sasahara, M. Hoshi, Y. Ishii, NMR-based site-resolved profiling of β-amyloid misfolding reveals structural transitions from pathologically relevant spherical oligomer to fibril. J. Biol. Chem. 295, 458-467 (2020).
31. K. A. Conway, J. D. Harper, P. T. Lansbury, Accelerated in vitro fibril formation by a mutant α-synuclein linked to early-onset Parkinson disease. Nat. Med. 4, 1318-1320 (1998).
32. M. Shahnawaz, A. Mukherjee, S. Pritzkow, N. Mendez, P. Rabadia, X. Liu, B. Hu, A. Schmeichel, W. Singer, G. Wu, A. L. Tsai, H. Shirani, K. P. R. Nilsson, P. A. Low, C. Soto, Discriminating α-synuclein strains in Parkinson's disease and multiple system atrophy. Nature 578, 273-277 (2020).
33. J. M. Tennant, D. M. Henderson, T. M. Wisniewski, E. A. Hoover, R T-QuIC detection of tauopathies using full-length tau substrates. Prion. 14, 249-256 (2020).
34. C. Koch, A. E. Posch, H. C. Goicoechea, C. Herwig, B. Lendl, Multi-analyte quantification in bioprocesses by Fourier-transform-infrared spectroscopy by partial least squares regression and multivariate curve resolution. Anal. Chim. Acta 807, 103-110 (2014).
35. M. Paraskevaidi, C. L. M. Morais, K. M. G. Lima, J. S. Snowden, J. A. Saxon, A. M. T. Richardson, M. Jones, D. M. A. Mann, D. Allsop, P. L. Martin-Hirsch, F. L. Martin, Differential diagnosis of Alzheimer's disease using spectrochemical analysis of blood. Proc. Natl. Acad. Sci. U.S.A. 114, E7929-E7938 (2017).
36. K. Araki, N. Yagi, Y. Ikemoto, H. Yagi, C. J. Choong, H. Hayakawa, G. Beck, H. Sumi, H. Fujimura, T. Moriwaki, Y. Nagai, Y. Goto, H. Mochizuki, Synchrotron FTIR micro-spectroscopy for structural analysis of Lewy bodies in the brain of Parkinson's disease patients. Sci. Rep. 5, 17625 (2015).
37. A. Sevinc, D. Yonar, F. Severcan, Investigation of neurodegenerative diseases from body fluid samples using Fourier transform infrared spectroscopy. Biomed. Spectrosc. Imaging 4, 341-357 (2015).
38. A. Barth, Infrared spectroscopy of proteins. Biochim. Biophys. Acta 1767, 1073-1101 (2007).
39. J. Kong, S. Yu, Fourier transform infrared spectroscopic analysis of protein secondary structures. Acta Biochim. Biophys. Sin. 39, 549-559 (2007).
40. A. Nabers, J. Ollesch, J. Schartner, C. Kötting, J. Genius, U. Haußmann, H. Klafki, J. Wiltfang, K. Gerwert, An infrared sensor analysing label-free the secondary structure of the Abeta peptide in presence of complex fluids. J. Biophotonics 9, 224-234 (2016).
41. A. Nabers, L. Perna, J. Lange, U. Mons, J. Schartner, J. Güldenhaupt, K. U. Saum, S. Janelidze, B. Holleczek, D. Rujescu, O. Hansson, K. Gerwert, H. Brenner, Amyloid blood biomarker detects Alzheimer's disease. EMBO Mol. Med. 10, e8763 (2018).
42. A. Nabers, J. Ollesch, J. Schartner, C. Kötting, J. Genius, H. Hafermann, H. Klafki, K. Gerwert, J. Wiltfang, Amyloid-β-secondary structure distribution in cerebrospinal fluid and blood measured by an immuno-infrared-sensor: A biomarker candidate for Alzheimer's disease. Anal. Chem. 88, 2755-2762 (2016).
43. L. Beyer, R. Günther, J. C. Koch, S. Klebe, T. Hagenacker, P. Lingor, A. S. Biesalski, A. Hermann, A. Nabers, R. Gold, L. Tönges, K. Gerwert, TDP-43 as structure-based biomarker in amyotrophic lateral sclerosis. Ann. Clin. Transl. Neurol. 8, 271-277 (2021).
44. H. Altug, S. H. Oh, S. A. Maier, J. Homola, Advances and applications of nanophotonic biosensors. Nat. Nanotechnol. 17, 5-16 (2022).
45. F. Neubrech, C. Huck, K. Weber, A. Pucci, H. Giessen, Surface-enhanced infrared spectro-scopy using resonant nanoantennas. Chem. Rev. 117, 5110-5145 (2017).
46. R. Adato, H. Altug, In-situ ultra-sensitive infrared absorption spectroscopy of biomolecule interactions in real time with plasmonic nanoantennas. Nat. Commun. 4, 2154 (2013).
47. D. Etezadi, J. B. Warner Iv, F. S. Ruggeri, G. Dietler, H. A. Lashuel, H. Altug, Nanoplasmonic mid-infrared biosensor for in vitro protein secondary structure detection. Light Sci. Appl. 6, e17029 (2017).
48. R. Adato, A. A. Yanik, J. J. Amsden, D. L. Kaplan, F. G. Omenetto, M. K. Hong, S. Erramilli, H. Altug, Ultra-sensitive vibrational spectroscopy of protein monolayers with plasmonic nanoantenna arrays. Proc. Natl. Acad. Sci. U.S.A. 106, 19227-19232 (2009).
49. R. Semenyshyn, M. Hentschel, C. Stanglmair, T. Teutsch, C. Tarin, C. Pacholski, H. Giessen, F. Neubrech, In vitro monitoring conformational changes of polypeptide monolayers using infrared plasmonic nanoantennas. Nano Lett. 19, 1-7 (2019).
50. A. John-Herpin, A. Tittl, H. Altug, Quantifying the limits of detection of surface-enhanced infrared spectroscopy with grating order-coupled nanogap antennas. ACS Photonics 5, 4117-4124 (2018).
51. R. Semenyshyn, F. Morz, T. Steinle, M. Ubl, M. Hentschel, F. Neubrech, H. Giessen, Pushing down the limit: In vitro detection of a polypeptide monolayer on a single infrared resonant nanoantenna. ACS Photonics 6, 2636-2642 (2019).
52. D. Etezadi, J. B. Warner, H. A. Lashuel, H. Altug, Real-time in situ secondary structure analysis of protein monolayer with mid-infrared plasmonic nanoantennas. ACS Sens. 3, 1109-1117 (2018).
53. S. T. Kumar, S. Donzelli, A. Chiki, M. M. K. Syed, H. A. Lashuel, A simple, versatile and robust centrifugation-based filtration protocol for the isolation and quantification of α-synuclein monomers, oligomers and fibrils: Towards improving experimental reproducibility in α-synuclein research. J. Neurochem. 153, 103-119 (2020).
54. L. C. Serpell, J. Berriman, R. Jakes, M. Goedert, R. A. Crowther, Fiber diffraction of synthetic α-synuclein filaments shows amyloid-like cross-β conformation. Proc. Natl. Acad. Sci. 97, 4897-4902 (2000).
55. M. F. Altay, S. T. Kumar, J. Burtscher, S. Jagannath, C. Strand, Y. Miki, L. Parkkinen, J. L. Holton, H. A. Lashuel, Development and validation of an expanded antibody toolset that captures alpha-synuclein pathological diversity in Lewy body diseases. bioRxiv 2022.05.26.493598 (2022). https://doi.org/10.1101/2022.05.26.493598.
56. H. Yang, S. Yang, J. Kong, A. Dong, S. Yu, Obtaining information about protein secondary structures in aqueous solution using Fourier transform IR spectroscopy. Nat. Protoc. 10, 382-396 (2015).
57. B. Winner, R. Jappelli, S. K. Maji, P. A. Desplats, L. Boyer, S. Aigner, C. Hetzer, T. Loher, M. Vilar, S. Campioni, C. Tzitzilonis, A. Soragni, S. Jessberger, H. Mira, A. Consiglio, E. Pham, E. Masliah, F. H. Gage, R. Riek, In vivo demonstration that α-synuclein oligomers are toxic. Proc. Natl. Acad. Sci. U.S.A. 108, 4194-4199 (2011).
58. K. M. Danzer, S. K. Krebs, M. Wolff, G. Birk, B. Hengerer, Seeding induced by α-synuclein oligomers provides evidence for spreading of α-synuclein pathology. J. Neurochem. 111, 192-203 (2009).
59. J. L. Lansford, D. G. Vlachos, Infrared spectroscopy data- and physics-driven machine learning for characterizing surface microstructure of complex materials. Nat. Commun. 11, 1513 (2020).
60. S. Zinchik, S. Jiang, S. Friis, F. Long, L. Høgstedt, V. M. Zavala, E. Bar-Ziv, Accurate characterization of mixed plastic waste using machine learning and fast infrared spectroscopy. ACS Sustainable Chem. Eng. 9, 14143-14151 (2021).
61. S. Jiang, Z. Xu, M. Kamran, S. Zinchik, S. Paheding, A. McDonald, E. Bar-Ziv, V. M. Zavala, Using ATR-FTIR spectra and convolutional neural networks for characterizing mixed plastic waste. Comput. Chem. Eng. 155, 107547 (2021).
62. Z. Ren, Z. Zhang, J. Wei, B. Dong, C. Lee, Wavelength-multiplexed hook nanoantennas for machine learning enabled mid-infrared spectroscopy. Nat. Commun. 13, 3859 (2022).
63. J. Meng, J. J. Cadusch, K. B. Crozier, Plasmonic mid-infrared filter array-detector array chemical classifier based on machine learning. ACS Photonics 8, 648-657 (2021).
64. A. N. Dizaji, N. S. Ozek, A. Yilmaz, F. Aysin, M. Yilmaz, Gold nanorod arrays enable highly sensitive bacterial detection via surface-enhanced infrared absorption (SEIRA) spectro-scopy. Colloids Surf. B Biointerfaces 206, 111939 (2021).
65. G. Kelp, N. Arju, A. Lee, E. Esquivel, R. Delgado, Y. Yu, S. Dutta-Gupta, K. Sokolov, G. Shvets, Application of metasurface-enhanced infra-red spectroscopy to distinguish between normal and cancerous cell types. Analyst 144, 1115-1127 (2019).
66. D. Rodrigo, A. Tittl, N. Ait-Bouziad, A. John-Herpin, O. Limaj, C. Kelly, D. Yoo, N. J. Wittenberg, S. H. Oh, H. A. Lashuel, H. Altug, Resolving molecule-specific information in dynamic lipid membrane processes with multi-resonant infrared metasurfaces. Nat. Commun. 9, 2160 (2018).
67. A. John-Herpin, D. Kavungal, L. von Mücke, H. Altug, Infrared metasurface augmented by deep learning for monitoring dynamics between all major classes of biomolecules. Adv. Mater. 33, e2006054 (2021).
68. L. Pan, L. Meng, M. He, Z. Zhang, Tau in the pathophysiology of Parkinson's disease. J. Mol. Neurosci. 71, 2179-2191 (2021).
69. K. Arima, S. Hirai, N. Sunohara, K. Aoto, Y. Izumiyama, K. Uéda, K. Ikeda, M. Kawai, Cellular co-localization of phosphorylated tau- and NACP/α-synuclein-epitopes in Lewy bodies in sporadic Parkinson's disease and in dementia with Lewy bodies. Brain Res. 843, 53-61 (1999).
70. J. H. Kang, D. J. Irwin, A. S. Chen-Plotkin, A. Siderowf, C. Caspell, C. S. Coffey, T. Waligórska, P. Taylor, S. Pan, M. Frasier, K. Marek, K. Kieburtz, D. Jennings, T. Simuni, C. M. Tanner, A. Singleton, A. W. Toga, S. Chowdhury, B. Mollenhauer, J. Q. Trojanowski, L. M. Shaw, Parkinson's Progression Markers Initiative, Association of cerebrospinal fluid β-amyloid 1-42, T-tau, P-tau181, and α-synuclein levels with clinical features of drug-naive patients with early Parkinson disease. JAMA Neurol. 70, 1277-1287 (2013).
71. J. H. Kang, B. Mollenhauer, C. S. Coffey, J. B. Toledo, D. Weintraub, D. R. Galasko, D. J. Irwin, V. Van Deerlin, A. S. Chen-Plotkin, C. Caspell-Garcia, T. Waligórska, P. Taylor, N. Shah, S. Pan, P. Zero, M. Frasier, K. Marek, K. Kieburtz, D. Jennings, C. M. Tanner, T. Simuni, A. Singleton, A. W. Toga, S. Chowdhury, J. Q. Trojanowski, L. M. Shaw; Parkinson's Progression Marker Initiative, CSF biomarkers associated with disease heterogeneity in early Parkinson's disease: The Parkinson's Progression Markers Initiative study. Acta Neuropathol. 131, 935-949 (2016).
72. A. Nabers, H. Hafermann, J. Wiltfang, K. Gerwert, Aβ and tau structure-based biomarkers for a blood- and CSF-based two-step recruitment strategy to identify patients with dementia due to Alzheimer's disease. Alzheimers Dement. (Amst). 11, 257-263 (2019).
73. B. Budde, J. Schartner, L. Tönges, C. Kötting, A. Nabers, K. Gerwert, Reversible immuno-infrared sensor for the detection of Alzheimer's disease related biomarkers. ACS Sens. 4, 1851-1856 (2019).
74. A. Belushkin, F. Yesilkoy, J. J. González-López, J. C. Ruiz-Rodríguez, R. Ferrer, A. Fàbrega, H. Altug, Rapid and digital detection of inflammatory biomarkers enabled by a novel portable nanoplasmonic imager. Small 16, 1906108 (2020).
75. A. Bouvier-Müller, F. Ducongé, Nucleic acid aptamers for neurodegenerative diseases. Biochimie 145, 73-83 (2018).
76. M. Rodrigues, P. Bhattacharjee, A. Brinkmalm, D. T. Do, C. M. Pearson, S. De, A. Ponjavic, J. A. Varela, K. Kulenkampff, I. Baudrexel, D. Emin, F. S. Ruggeri, J. E. Lee, A. R. Carr, T. P. J. Knowles, H. Zetterberg, T. N. Snaddon, S. Gandhi, S. F. Lee, D. Klenerman, Structure-specific amyloid precipitation in biofluids. Nat. Chem. 14, 1045-1053 (2022).
77. T. Lee, C. Lee, D. K. Oh, T. Badloe, J. G. Ok, J. Rho, Scalable and high-throughput top-down manufacturing of optical metasurfaces. Sensors 20, 4108 (2020).
78. A. Leitis, M. L. Tseng, A. John-Herpin, Y. S. Kivshar, H. Altug, Wafer-scale functional metasurfaces for mid-infrared photonics and biosensing. Adv. Mater. 33, 2102232 (2021).
79. A. Tittl, A. Leitis, M. Liu, F. Yesilkoy, D. Y. Choi, D. N. Neshev, Y. S. Kivshar, H. Altug, Imaging-based molecular barcoding with pixelated dielectric metasurfaces. Science 360, 1105-1109 (2018).
80. B. Hinkov, F. Pilat, L. Lux, P. L. Souza, M. David, A. Schwaighofer, D. Ristanić, B. Schwarz, H. Detz, A. M. Andrews, B. Lendl, G. Strasser, A mid-infrared lab-on-a-chip for dynamic reaction monitoring. Nat. Commun.13,4753 (2022).
81. R. L. Olmon, B. Slovick, T. W. Johnson, D. Shelton, S. H. Oh, G. D. Boreman, M. B. Raschke, Optical dielectric function of gold. Phys. Rev. B 862012). 10.1103/PhysRevB.86.235147.
82. A. D. Rakić, A. B. Djurišić, J. M. Elazar, M. L. Majewski, Optical properties of metallic films for vertical-cavity optoelectronic devices. Appl. Opt. 37, 5271 (1998). 10.1364/A0.37.005271.
83. H. H. Li, Refractive index of alkaline earth halides and its wavelength and temperature derivatives. J. Phys. Chem. Ref. 9, 161-290 (1980). 10.1063/1.555616.
84. G. M. Hale, M. R. Querry, Optical Constants of Water in the 200-nm to 200-μm Wavelength Region. Appl. Opt. 12, 555 (1973). 10.1364/AO.12.000555.

1. A neurodegenerative disorder biosensing system including:
at least one plasmonic device including a plurality of plasmonic nanostructures configured to provide plasmonic excitation surface-enhanced infra-red absorption by molecular vibrational excitations of neurodegenerative disorder proteins, the plurality of plasmonic nanostructures being configured to have attached thereto capturing agents configured to bind to protein secondary structure types formed from neurodegenerative disorder aggregated proteins;
at least one optical detector configured to detect reflected optical infra-red spectra reflected from the plurality of plasmonic nanostructures of the at least one plasmonic device; and
at least one data processing device including at least one processor, comprising processing circuitry, individually and/or collectively configured to process a plurality of absorption spectra determined from the reflected optical infra-red spectra, the plurality of absorption spectra representing time-resolved infra-red absorption by at least one of: (i) first protein secondary structure types formed from neurodegenerative disorder aggregated proteins and (ii) second protein secondary structure types formed from neurodegenerative disorder aggregated proteins;
wherein the at least one processor of the at least one data processing device is individually and/or collectively configured to process the plurality of absorption spectrum signals to identify the first protein secondary structure type and the second protein secondary structure type, and to distinguish the identified first protein secondary structure type from the second protein secondary structure type, and to distinguish the identified second protein secondary structure type from the first protein secondary structure type, wherein the first protein secondary structure type is different to the second protein secondary structure type.

2. The neurodegenerative disorder biosensing system according to claim 1, the first protein secondary structure type and the second protein secondary structure type are evolving and distinct intermediate structural species of a neurodegenerative disorder protein-aggregation body formation process.

3. The neurodegenerative disorder biosensing system according to claim 1, wherein the plurality of absorption spectra represent time-resolved infra-red absorption by at least one of: (i) neurodegenerative disorder monomeric proteins, (ii) first protein secondary structure types formed from neurodegenerative disorder aggregated proteins and (iii) second protein secondary structure types formed from neurodegenerative disorder aggregated proteins; and wherein the at least one data processing device is configured to process the plurality of absorption spectrum signals to identify the neurodegenerative disorder monomeric protein, the first protein secondary structure type and the second protein secondary structure type, and to distinguish the identified first protein secondary structure type from the second protein secondary structure type, the identified second protein secondary structure type from the first protein secondary structure type and the identified neurodegenerative disorder monomeric protein from the first and second protein secondary structure types.

4. The neurodegenerative disorder biosensing system according to claim 1, wherein the at least one processor of the at least one data processing device is individually and/or collectively configured to process the plurality of absorption spectra to determine the simultaneous presence of the first protein secondary structure type and the second protein secondary structure type.

5. The neurodegenerative disorder biosensing system according to claim 1, wherein, in the performing of the identification, the at least one processor of the at least one data processing device is individually and/or collectively configured to process the plurality of absorption spectra to determine distinguishing contributions to the absorption spectra by a secondary structure of the first or second protein secondary structure types formed from aggregated proteins.

6. The neurodegenerative disorder biosensing system according to claim 1, wherein the at least one processor of the at least one data processing device is individually and/or collectively configured to distinguish the first protein secondary structure type from the second protein secondary structure type and the second protein secondary structure type from the first protein secondary structure type by determining a relative absorption contribution of a plurality of different constituent secondary structure structural motifs.

7. The neurodegenerative disorder biosensing system according to claim 6, wherein the constituent secondary structure structural motifs include at least one of: alpha helices, beta-sheets and beta-turns.

8. The neurodegenerative disorder biosensing system according to claim 1, wherein the at least one processor of the at least one data processing device is individually and/or collectively configured to process the plurality of absorption spectra to determine a rate of change of a rate of change of an absorption value over a spectral range of the absorption spectra.

9. The neurodegenerative disorder biosensing system according to claim 1, wherein the at least one processor of the at least one data processing device is individually and/or collectively configured to process the plurality of absorption spectra to deconvolute absorption contributions by a plurality of different constituent secondary structure structural motifs.

10. The neurodegenerative disorder biosensor system according to claim 1, wherein the at least one processor of the at least one data processing device is individually and/or collectively configured to process at least one absorption spectrum to determine a quantity ratio of the first protein secondary structure type to the second protein secondary structure type.

11. The neurodegenerative disorder biosensing system according to claim 10, including a trained deep neural network configured to process at least one absorption spectra to determine the quantity ratio of first protein secondary structure type to the second protein secondary structure type quantity ratio when provided with at least one absorption spectrum inputted to the trained deep neural network.

12. The neurodegenerative disorder biosensing system according to claim 10, wherein the first protein secondary structure type to second protein secondary structure type quantity ratio is an oligomer to Fibril quantity ratio.

13. The neurodegenerative disorder biosensing system according to claim 1, including at least one microfluidic device comprising at least one microfluidic channel configured to communicate at least one fluid to the plurality of plasmonic nanostructures from at least one fluid inlet for immunoassay measurements, the at least one microfluidic device including the at least one plasmonic device such that the at least one plasmonic device defines a portion of the at least one microfluidic channel.

14. The neurodegenerative disorder biosensing system according to claim 13, wherein the at least one microfluidic device includes a plurality of microfluidic channels to permit multiplexed neurodegenerative disorder protein detection of different neurodegenerative disorder proteins, each microfluidic channel is configured to communicate at least one fluid to at least one sensing element comprising a plurality of plasmonic nanostructures, the at least one plasmonic device including the sensing elements each comprising a plurality of plasmonic nanostructures, wherein each sensing element defines a portion of the microfluidic channel.

15. The neurodegenerative disorder biosensing system according to claim 1, including at least one microfluidic device, wherein the at least one plasmonic device includes a plurality of microfluidic wells and a plurality of sensing elements each sensing element comprising a plurality of plasmonic nanostructures, and wherein each microwell includes at least one sensing element to capture neurodegenerative disorder proteins of the microwell.

16. The neurodegenerative disorder biosensing system according to claim 1, wherein the first protein secondary structure type is an alpha-synuclein oligomer, and the second protein secondary structure type is an alpha-synuclein fibril.

17. A neurodegenerative disorder biosensing method including:
providing at least one plasmonic device including a plurality of plasmonic nanostructures configured to provide plasmonic excitation surface-enhanced infra-red absorption by molecular vibrational excitations of neurodegenerative disorder proteins, the plurality of plasmonic nanostructures having attached thereto capturing agents configured to bind to protein secondary structure types formed from neurodegenerative disorder aggregated proteins;
providing at least one optical detector configured to detect reflected optical infra-red spectra reflected from the plurality of plasmonic nanostructures of the at least one plasmonic device;
providing at least one fluidic sample to the at least one plasmonic device;
determining a plurality of absorption spectra from obtained reflected optical infra-red spectra, the plurality of absorption spectra representing time-resolved infra-red absorption by at least one of: (i) first protein secondary structure types formed from neurodegenerative disorder aggregated proteins and (ii) second protein secondary structure types formed from neurodegenerative disorder aggregated proteins;
identifying, from the plurality of absorption spectra, at least one the first protein secondary structure type and the second protein secondary structure type, and
distinguishing the identified first protein secondary structure type from the second protein secondary structure type and the identified second protein secondary structure type from the first protein secondary structure type, wherein the first protein secondary structure type is different to the second protein secondary structure type.

18. The neurodegenerative disorder biosensing method according to claim 17, wherein the plurality of plasmonic nanostructures have attached thereto capturing agents configured to bind to neurodegenerative disorder monomeric proteins, and the plurality of absorption spectra represent time-resolved infra-red absorption by at least one of: (i) neurodegenerative disorder monomeric proteins, (ii) first protein secondary structure types formed from neurodegenerative disorder aggregated proteins and (iii) second protein secondary structure types formed from neurodegenerative disorder aggregated proteins; and the distinguishing step includes distinguishing the identified neurodegenerative disorder monomeric protein from the first and second protein secondary structure types.

19. The neurodegenerative disorder biosensing method according to claim 17, wherein the first and second protein secondary structure types are evolving and distinct intermediate structural species of a neurodegenerative disorder protein-aggregation body formation process.

20. The neurodegenerative disorder biosensing method according to claim 17, wherein the distinguishing of the first protein secondary structure type from the second protein secondary structure type, the second protein secondary structure type from the first protein secondary structure type is by determining a relative absorption contribution of a plurality of different constituent secondary structure structural motifs.

21. The neurodegenerative disorder biosensing method according to claim 17, including determining a first protein secondary structure type to second protein secondary structure type quantity ratio by inputting at least one absorption spectrum to a trained deep neural network configured to process at least one absorption spectrum to determine a first protein secondary structure type to second protein secondary structure type quantity ratio.

22. A non-transitory computer readable medium having computer code recorded thereon, the computer code configured to perform a neurodegenerative disorder biosensing method when executed on at least one data processing device comprising processing circuitry of a computer device, the neurodegenerative disorder biosensing method comprising:
processing a plurality of absorption spectra determined from reflected optical infra-red spectra, the plurality of absorption spectra representing time-resolved infra-red absorption by at least one of: (i) first protein secondary structure types formed from neurodegenerative disorder aggregated proteins and (ii) second protein secondary structure types formed from neurodegenerative disorder aggregated proteins;
processing the plurality of absorption spectra to identify at least one of the first protein secondary structure type and the second protein secondary structure type and to distinguish the identified first protein secondary structure type from the second protein secondary structure type and the identified second protein secondary structure type from the first protein secondary structure type.
